(19) United States
(12) Patent Application Publication
(10) Pub. No.: US 2011/0224176 A1
(43) Pub. Date: Sep. 15, 2011

(54) Lyophilized Cake Formulations

(75) Inventors: John Daniel Dobak, La Jolla, CA (US); Chris Kemmerer, San Diego, CA (US); Kenneth Walter Locke, Carlsbad, CA (US)

(73) Assignee: Lithera, Inc., San Diego, CA (US)

(21) Appl. No.: 13/007,518

(22) Filed: Jan. 14, 2011

Related U.S. Application Data
(60) Provisional application No. 61/295,646, filed on Jan. 15, 2010.

(51) Int. Cl. A61K 31/56; A61K 31/138; A61P 3/04; A61P 9/00; A61P 3/10

(52) U.S. Cl. 514/171; 514/180; 514/651

(57) ABSTRACT

Provided herein are lyophilized cake forms of fluticasone, salmeterol, or a pharmaceutically acceptable salt or a combination thereof which provides room temperature stability for an extended period of time. Upon reconstitution with an acceptable solvent (e.g., a carrier or diluent), the reconstituted pharmaceutical or cosmetic formulation provides a sterile, non-suspension form suitable for parenteral injectable administration, including subcutaneous injection.

### CROSS REFERENCE

This application claims the benefit of U.S. Appl. No. 61/295,646 filed Jan. 15, 2010 which is hereby incorporated by reference in its entirety.

### BACKGROUND OF THE INVENTION

Excess body fat is a severe health care issue in modern societies. Chronic health conditions promoted by excess body fat include, e.g., cardiovascular disease and diabetes mellitus type 2. Moreover, excess body fat greatly undermines personal appearance and self image. Long acting beta adrenergic receptor agonists and/or corticosteroids are used in injectable pharmaceutical preparations to reduce excess body fat.

Lyophilized products, such as injectable pharmaceutical preparations, which are relatively unstable in aqueous solution can result in solid phase products that are more stable and therefore have a longer shelf life.

### SUMMARY OF THE INVENTION

Provided herein are lyophilized cake compositions comprising a lyophilized active ingredient such as by way of example only, salmeterol xinafoate, fluticasone propionate, other pharmaceutically acceptable salts or a combination and mixtures thereof and a bulking agent. In some embodiments, the lyophilized compositions further comprise a non-ionic surfactant. In further or additional embodiments, the lyophilized compositions comprise a buffering agent. In still further or additional embodiments, the lyophilized compositions described herein further comprise an anti-oxidant. The lyophilized compositions described herein are prepared from a bulk solution which, in some embodiments, is sterilized via filtration methods prior to lyophilization. The lyophilized cake compositions provide a stable form of active ingredient which, in other embodiments, is easily reconstitutable prior to administration. The present application also describes methods of preparing such lyophilized cake compositions, formulations comprising reconstituted lyophile formulations, methods of preparing reconstituted lyophile formulations, and methods of using lyophilized compositions and reconstituted lyophile formulations for therapeutic and cosmetic indications.

### Lyophilized Fluticasone Compositions

In a first aspect, provided herein are sterile fluticasone lyophile compositions comprising lyophilized fluticasone or a pharmaceutically acceptable salt thereof, a bulking agent and a non-ionic surfactant. In some embodiments, the sterile fluticasone lyophile further comprises a buffering agent. In some embodiments, the buffering agent is citric acid or sodium citrate dihydrate. In one embodiment, the buffering agent is citric acid. In another embodiment, the buffering agent is citrate dihydrate. Also described herein are sterile lyophile compositions comprising lyophilized fluticasone in a crystalline phase, an amorphous phase, a semi-crystalline phase, a semi-amorphous phase, or a mixture thereof. In one embodiment, the lyophilized fluticasone is in an amorphous phase. In another embodiment, the pharmaceutically acceptable salt of fluticasone is a propionate salt or a furoate salt. In yet another embodiment, the pharmaceutically acceptable salt is the propionate salt.

In some embodiments, provided is a sterile lyophile fluticasone composition wherein the lyophilized fluticasone is present in an amount of about 0.001% to about 0.1% by weight of the total combined dry weight of the lyophile. In yet a further embodiment, the lyophilized fluticasone is present in an amount of about 0.015% to about 0.030% by weight of the total combined dry weight of the lyophile. In yet a further embodiment, the lyophilized fluticasone is present in an amount of about 0.017% to about 0.023% by weight of the total combined dry weight of the lyophile In one embodiment the lyophilized fluticasone is present in an amount of about 0.020% by weight of the total combined dry weight of the lyophile.

In further or additional embodiments, provided herein are sterile lyophile fluticasone compositions comprising lyophilized fluticasone that is present in an amount equal to or less than about 500 micrograms/gram. In some embodiments, provided is a sterile lyophile fluticasone composition wherein the lyophilized fluticasone is present in an amount of about 1 microgram/gram to about 500 micrograms/gram. In further or additional embodiments, provided is a sterile lyophile fluticasone composition wherein the lyophilized fluticasone is present in an amount of about 5 micrograms/gram to about 350 micrograms/gram. In certain embodiments, provided is a sterile lyophile fluticasone composition wherein the lyophilized fluticasone is present in an amount of about 15 micrograms/gram to about 300 micrograms/gram. In some embodiments, provided herein are sterile fluticasone compositions wherein the lyophilized fluticasone is present in an amount of about 20 micrograms/gram to about 250 micrograms/gram. In yet additional embodiments, provided is a sterile lyophilized fluticasone formulation wherein the lyophilized fluticasone is present in an amount of about 25 micrograms/gram to about 225 micrograms/gram. In certain embodiments, provided herein are sterile lyophile fluticasone compositions wherein the lyophilized fluticasone is present in an amount of about 30 micrograms/gram to about 200 micrograms/gram. In yet further or additional embodiments, described herein are sterile lyophilized fluticasone compositions comprising about 100 micrograms/gram to about 300 micrograms/gram of lyophilized fluticasone. In some embodiments, provided herein are sterile lyophilized fluticasone formulations comprising about 200 micrograms/gram to about 300 micrograms/gram. In one embodiment, provided herein are sterile lyophilized fluticasone compositions comprising about 150 micrograms/gram of lyophilized fluticasone.

Also provided herein are sterile lyophilized fluticasone compositions comprising a lyophile of fluticasone that is present in an amount that is equal to or less than about 50 micrograms/gram. In further or additional embodiments, provided herein is a sterile lyophile fluticasone composition wherein the lyophilized fluticasone is present in an amount of about 1 microgram/gram to about 50 micrograms/gram. In certain embodiments, provided is a sterile lyophile fluticasone composition wherein the lyophilized fluticasone is present in an amount of about 5 micrograms/gram to about 35 micrograms/gram. In one embodiment, provided is a sterile lyophile fluticasone composition wherein the lyophilized fluticasone is present in an amount of about 9 micrograms/gram to about 20 micrograms/gram.

Also described herein, in further or additional embodiments, is a sterile fluticasone lyophile composition comprising lyophilized fluticasone or a pharmaceutically acceptable salt thereof, a bulking agent and a non-ionic surfactant, such as by way of example only, polysorbate 80. In some embodiments, the non-ionic surfactant is selected from (N,N-Bis[3-(D-gluconamido)propyl]cholamide); Bis(polyethylene glycol bis[imidazoyl carbonyl]); Polyoxyethyleneglycol dodecyl ether; Polyoxyethylenglyceroltriricinoleate 35; decaethylene glycol monododecyl ether; N-decanoyl-N-methylglucamine; n-decyl α-D-glucopyranoside; decyl β-D-maltopyranoside; n-dodecanoyl-N-methylglucamide; n-dodecyl α-D-maltoside; n-dodecyl β-D-maltoside; n-hexadecyl β-D-maltoside; heptaethylene glycol monodecyl ether; heptaethylene glycol monododecyl ether; heptaethylene glycol monotetradecyl ether; hexaethylene glycol monododecyl ether; hexaethylene glycol monohexadecyl ether; hexaethylene glycol monooctadecyl ether; hexaethylene glycol monotetradecyl ether; octylphenyl-polyethylene glycol; methyl-6-O-(N-heptylcarbamoyl)-α-D-glucopyranoside; nonaethylene glycol monododecyl ether; N-nonanoyl-N-methylglucamine; N-nonanoyl-N-methylglucamine; octaethylene glycol monodecyl ether; octaethylene glycol monododecyl ether; octaethylene glycol monohexadecyl ether; octaethylene glycol monooctadecyl ether; octaethylene glycol monotetradecyl ether; octyl-β-D-glucopyranoside; pentaethylene glycol monodecyl ether; pentaethylene glycol monododecyl ether; pentaethylene glycol monohexadecyl ether; pentaethylene glycol monohexyl ether; pentaethylene glycol monooctadecyl ether; pentaethylene glycol monooctyl ether; polyethylene glycol diglycidyl ether; polyethylene glycol ether W-1; polyoxyethylene 10 tridecyl ether; polyoxyethylene 100 stearate; polyoxyethylene 20 isohexadecyl ether; polyoxyethylene 20 oleyl ether; polyoxyethylene 40 stearate; polyoxyethylene 50 stearate; polyoxyethylene 8 stearate, polyoxyethylene bis(imidazolyl carbonyl), polyoxyethylene 25 propylene glycol stearate; saponin; sorbitan laurate, sorbitan monopalmitate, sorbitan stearate, sorbitan tristearate, sorbitan oleate, sorbitan trioleate; polyethyelene glycol trimethylnonyl ether, nonylphenol ethoxylate; tetradecyl-β-D-maltoside; tetraethylene glycol monodecyl ether, tetraethylene glycol monododecyl ether, tetraethylene glycol monotetradecyl ether; triethylene glycol monodecyl ether, triethylene glycol monododecyl ether, triethylene glycol monohexadecyl ether, triethylene glycol monooctyl ether, triethylene glycol monotetradecyl ether; octoxynol-9; octylphenol ethoxylate; polysorbate 20, polysorbate 21, polysorbate 40, polysorbate 60, polysorbate 61, polysorbate 65, polysorbate 80, polysorbate 81, polysorbate 85; tyloxapol; and n-undecyl β-D-glucopyranoside. In a specific embodiment, the lyophile fluticasone composition comprises a non-ionic surfactant that is polysorbate 80.

In one embodiment, the non-ionic surfactant is present in amount of about 1% to about 10% by weight of the total combined dry weight of the lyophile. In another embodiment, the non-ionic surfactant is present in an amount of about 2.5% to about 7.5% by weight of the total combined dry weight of the lyophile. In another embodiment, the non-ionic surfactant is present in an amount of about 4.2% to about 5.8% by weight of the total combined dry weight of the lyophile. In a further embodiment, the non-ionic surfactant is present in an amount of about 5.0% by weight of the total combined dry weight of the lyophile.

In yet a further embodiment, provided is a sterile lyophile fluticasone composition wherein the bulking agent is selected from the group consisting of lactose, mannitol, dextrose, and sucrose. In another embodiment, the bulking agent is lactose monohydrate. In one embodiment, the bulking agent is present in an amount of about 90% to about 99% by weight of the total combined dry weight of the lyophile. In another embodiment, the bulking agent is present in an amount of about 93% to about 97% by weight of the total combined dry weight of the lyophile. In yet another embodiment, the bulking agent is present in an amount of about 95% by weight of the total combined dry weight of the lyophile.

In some embodiments, provided is a sterile lyophile fluticasone composition that comprises a non-ionic surfactant that is present in an amount of about 1% to about 10% by weight of the total combined dry weight of the lyophile and/or the bulking agent is present in an amount of about 90% to about 99% by weight of the total combined dry weight of the lyophile. In further or additional embodiments, the lyophile fluticasone composition comprises a non-ionic surfactant that is present in an amount of about 2.5% to about 7.5% by weight of the total combined dry weight of the lyophile and/or the bulking agent is present in an amount of about 93% to about 97% by weight of the total combined dry weight of the lyophile. In some embodiments, the lyophile fluticasone comprises a non-ionic surfactant that is present in an amount of about 4.2% to about 5.8% by weight of the total combined dry weight of the lyophile and/or the bulking agent is present in an amount of about 95% by weight of the total combined dry weight of the lyophile. In yet further or additional embodiments, provided is a lyophile fluticasone composition that comprises a non-ionic surfactant that is present in an amount of about 3.5% to about 5% by weight of the total combined dry weight of the lyophile and/or the bulking agent is present in an amount of at least about 95% by weight of the total combined dry weight of the lyophile.

In one embodiment, provided is a sterile lyophile fluticasone composition that is stable for a period of at least 4 weeks at a temperature of about 0° C. to about 50° C. In another embodiment, the fluticasone lyophile composition is stable for a period of at least 6 months at a temperature of about 0° C. to about 50° C.

In another embodiment, also provided herein are sterile lyophilized fluticasone compositions that, upon reconstitution with an aqueous solution, are suitable for subcutaneous, peri-orbital, intra-orbital, and intramuscular administration. In one embodiment, the fluticasone lyophile composition is suitable, upon reconstitution with an aqueous solution, for subcutaneous administration.

In one embodiment, the sterile lyophile fluticasone composition contains less than about 3% solvent or co-solvent by weight of the total combined dry weight of the lyophile. In another embodiment, the composition contains less than about 2% solvent or co-solvent by weight of the total combined dry weight of the lyophile. In a further embodiment, the composition contains less than about 1% solvent or co-solvent by weight of the total combined dry weight of the lyophile. In yet a further embodiment, the solvent or co-solvent is selected from tert-butyl alcohol, n-butanol, ethanol, iso-propyl alcohol, dimethyl sulfone, chlorobutanol, or mixtures thereof. In yet another embodiment, the composition contains less than about 2% water by weight of the total combined dry weight of the lyophile.

Reconstituted Fluticasone Formulations

In a second aspect, provided herein is a sterile fluticasone formulation that has been reconstituted from a lyophilized composition that comprised lyophilized fluticasone or a pharmaceutically acceptable salt thereof, a bulking agent and a non-ionic surfactant. In some embodiments, the sterile fluticasone lyophile further comprised a buffering agent. In some embodiments, the buffering agent is citric acid or sodium citrate dihydrate. In one embodiment, the buffering agent is citric acid. In another embodiment, the buffering agent is citrate dihydrate. In some embodiments, the reconstituted sterile fluticasone formulation further comprises a pharmaceutically acceptable diluent. In some embodiments, the diluent is selected from Sterile Water for Injection, 0.9% sodium chloride solution, or 5% dextrose solution. In a specific embodiment, the pharmaceutically acceptable diluent is Sterile Water for Injection.

In some embodiments, provided is a reconstituted sterile fluticasone formulation wherein the reconstituted fluticasone is present in an amount that is equal to or less than about 100 micrograms/milliliter. In further or additional embodiments, the reconstituted sterile fluticasone formulation comprises reconstituted fluticasone that is present in an amount of about 100 nanograms/milliliter to about 100 micrograms/milliliter. In some embodiments, the reconstituted sterile fluticasone formulation comprises reconstituted fluticasone that is present in an amount of about 200 nanograms/milliliter to about 50 micrograms/milliliter. In further or additional embodiments, provided is a sterile fluticasone formulation that comprises reconstituted fluticasone that is present in an amount of about 300 nanograms/milliliter to about 25 micrograms/milliliter. In yet further or additional embodiments, provided is a sterile fluticasone formulation that comprises reconstituted fluticasone that is present in an amount of about 500 nanograms/milliliter to about 15 micrograms/milliliter. In some embodiments, provided is a sterile fluticasone formulation comprising reconstituted fluticasone that is present in an amount of about 600 nanograms/milliliter to about 10 micrograms/milliliter. In some embodiments, provided is a sterile fluticasone formulation that comprises reconstituted fluticasone that is present in an amount of about 750 nanograms/milliliter to about 5 micrograms/milliliter. In some embodiments, provided is a reconstituted fluticasone formulation that comprises about 800 nanograms/milliliter to about 3 micrograms/milliliter of fluticasone or a pharmaceutically acceptable salt thereof. In yet further or additional embodiments, provided is a sterile fluticasone formulation comprising reconstituted fluticasone that is present in an amount of about 900 nanograms/milliliter to about 2 micrograms/milliliter. In a specific embodiment, provided is a reconstituted sterile fluticasone formulation comprising reconstituted fluticasone that is present in an amount of about 1 microgram/milliliter.

Described herein, in certain embodiments, is a reconstituted sterile fluticasone formulation from a lyophile fluticasone composition that comprised lyophilized fluticasone or a pharmaceutically acceptable salt thereof, a bulking agent and a non-ionic surfactant and a pharmaceutically acceptable diluent, wherein the reconstituted sterile fluticasone formulation is formulated for subcutaneous, peri-orbital, intra-orbital, and intramuscular injection. In another embodiment, the reconstituted sterile fluticasone formulation is formulated for subcutaneous administration, such as, for example, subcutaneous injection. In one embodiment, the lyophile is dissolved in the pharmaceutically acceptable diluent. In another embodiment, the dissolution occurs in less than about 2 minutes. In a further embodiment, the dissolution occurs in about 1 minute. In yet a further embodiment, dissolution occurs in less than about 1 minute. In yet another embodiment, dissolution occurs in less than about 30 seconds. In yet a further embodiment, dissolution results in a substantially clear solution.

In some embodiments, provided is a reconstituted lyophile fluticasone formulation that provides a solubilized or dissolved amount of fluticasone that is at least about 50% of the amount of the lyophilized fluticasone present prior to reconstitution as determined by HPLC after about 2 minutes of dissolution. In further or additional embodiments, provided herein is a reconstituted lyophile fluticasone formulation that provides a solubilized or dissolved amount of fluticasone that is at least about 60% of the amount of lyophilized fluticasone present prior to reconstitution as determined by HPLC after about 2 minutes of dissolution. In some embodiments, provided is a reconstituted lyophile fluticasone formulation that provides a solubilized or dissolved amount of fluticasone that is at least about 75% of the amount of lyophilized fluticasone present prior to reconstitution as determined by HPLC after about 2 minutes of dissolution. In further or additional embodiments, provided is a reconstituted lyophile fluticasone formulation that provides a solubilized or dissolved amount of fluticasone that is about 90% to about 100% of the amount of lyophilized fluticasone present prior to reconstitution as determined by HPLC after about 2 minutes of dissolution. In some embodiments, provided is a reconstituted lyophile fluticasone formulation that provides a solubilized or dissolved amount of fluticasone that is about 90% to about 100% of the amount of lyophilized fluticasone present prior to reconstitution as determined by HPLC after about 2 minutes of dissolution wherein the non-ionic surfactant was present in the lyophile fluticasone composition prior to reconstitution in an amount of about 4% to about 5% of the total combined dry weight of the lyophile.

Reconstituted Fluticasone Formulations Further Comprising Salmeterol

In a third aspect, provided herein is a sterile fluticasone formulation that has been reconstituted from a lyophilized composition that comprised lyophilized fluticasone or a pharmaceutically acceptable salt thereof, a bulking agent and a non-ionic surfactant wherein the sterile fluticasone formulation further comprises salmeterol. In some embodiments, the sterile fluticasone lyophile further comprised a buffering agent. In some embodiments, the buffering agent is citric acid or sodium citrate dihydrate. In one embodiment, the buffering agent is citric acid. In another embodiment, the buffering agent is citrate dihydrate. In some embodiments, the salmeterol has been reconstituted from a lyophile composition. In other embodiments, the salmeterol has not been reconstituted from a lyophile composition. In some embodiments, the reconstituted sterile fluticasone formulation further comprises a pharmaceutically acceptable diluents including as one example Sterile Water for Injection. In some embodiments, the sterile fluticasone formulation is formulated to further comprise salmeterol or a pharmaceutically acceptable salt thereof. In further or additional embodiments, the formulation further comprises a pharmaceutically acceptable diluent that is selected from Sterile Water for Injection, 0.9% sodium chloride solution, or 5% dextrose solution. In a specific embodiment, the pharmaceutically acceptable diluent is Sterile Water for Injection.

In some embodiments, provided herein is a sterile fluticasone formulation that has been reconstituted from a lyophilized composition that comprised lyophilized fluticasone or a pharmaceutically acceptable salt thereof, a bulking agent and a non-ionic surfactant wherein the sterile fluticasone formulation further comprises an amount that is equal to or less than about 25 micrograms/milliliter of salmeterol or a pharmaceutically acceptable salt thereof. In some embodiments, the reconstituted sterile fluticasone formulation further comprises salmeterol or a pharmaceutically acceptable salt thereof that is present in an amount that is equal to or less than about 1 microgram/milliliter. In further or additional embodiments, provided herein is a reconstituted sterile fluticasone formulation further comprising salmeterol that is present in an amount of about 2 nanograms/milliliter to about 25 micrograms/milliliter or about 1 microgram/milliliter. In further or additional embodiments, provided is a reconstituted sterile fluticasone formulation that further comprises salmeterol that is present in an amount of about 5 nanograms/milliliter to about 750 nanograms/milliliter. In yet additional embodiments, provided is a reconstituted sterile fluticasone formulation further comprising salmeterol or a pharmaceutically acceptable salt thereof that is present in an amount of about 10 nanograms/milliliter to about 500 nanograms/milliliter. In some embodiments, provided is a reconstituted sterile fluticasone formulation that further comprises salmeterol or a pharmaceutically acceptable salt thereof that is present in an amount of about 15 nanograms/milliliter to about 250 nanograms/milliliter.

In yet further or additional embodiments, the reconstituted sterile fluticasone formulation further comprises salmeterol or a pharmaceutically acceptable salt thereof that is present in an amount of about 15 nanograms/milliliter to about 50 nanograms/milliliter. In certain embodiments, provided is a reconstituted sterile fluticasone formulation that further comprises salmeterol or a pharmaceutically salt thereof wherein the salmeterol or salt is present in an amount of about 20 nanograms/milliliter to about 25 nanograms/milliliter. In a specific embodiment, the reconstituted sterile fluticasone formulation further comprises salmeterol or a pharmaceutically acceptable salt thereof that is present in an amount of about 20 nanograms/milliliter. In still further or additional embodiments, the reconstituted sterile fluticasone formulation further comprises salmeterol or a pharmaceutically acceptable salt thereof and about 1 microgram/milliliter of fluticasone or a pharmaceutically acceptable salt thereof. In certain embodiments, the reconstituted sterile fluticasone formulation further comprises salmeterol or a pharmaceutically acceptable salt thereof and is formulated for subcutaneous, peri-orbital, intra-orbital, and intramuscular injection. In further or additional embodiment, provided herein is a reconstituted sterile fluticasone formulation that further comprises salmeterol or a pharmaceutically acceptable salt thereof and is formulated for subcutaneous administration by injection.

Therapeutic and Cosmetic Methods of Using Fluticasone

In a fourth aspect, provided herein are cosmetic or therapeutic methods comprising administering or providing, including for example by subcutaneous administration, to a human a sterile fluticasone formulation that has been reconstituted from a lyophilized composition that comprised lyophilized fluticasone or a pharmaceutically acceptable salt thereof, a bulking agent and a non-ionic surfactant. In some embodiments, the sterile fluticasone lyophile further comprised a buffering agent. In some embodiments, the buffering agent is citric acid or sodium citrate dihydrate. In one embodiment, the buffering agent is citric acid. In another embodiment, the buffering agent is citrate dihydrate. In some embodiments, the method is therapeutic. In further or additional embodiments, the method is cosmetic.

In some embodiments, the reconstituted sterile fluticasone formulation is used (for example by provision or administration) to treat an indication selected from the group consisting of abdominal adiposity, regional adiposity, and exophthalmos due to thyroid eye disease. In certain embodiments, the reconstituted sterile fluticasone formulation is administered or provided subcutaneously. In some embodiments, the reconstituted sterile fluticasone formulation is administered or provided to the human subcutaneously as a peri-orbital or intra-orbital injection. In some embodiments, the reconstituted sterile fluticasone formulation is administered or provided to the human subcutaneously to an abdominal region or an ophthalmic region. In further or additional embodiments, provided is a reconstituted fluticasone formulation wherein the formulation is administered or provided to the human in the inside region of the knees, the middle to upper area of the upper arm (including the tricep area), the submental area (including the area under the chin, for example the wattle (which is understood to refer to the fleshy fold of skin in the submental area of a patient)), the abdomen, the hips, the inner thigh, the outer thigh, the buttocks, the lower back, the upper back or the chest.

In further embodiments, provided herein is a cosmetic method comprising administering or providing, including for example by subcutaneous administration, to a human a sterile fluticasone formulation that has been reconstituted from a lyophilized composition that comprised lyophilized fluticasone or a pharmaceutically acceptable salt thereof, a bulking agent and a non-ionic surfactant. In some embodiments, the reconstituted sterile fluticasone formulation is provided cosmetically to the human to affect a shape, contour, or appearance of the human body. In further or additional embodiments, the shape, contour, or appearance is in a region of the body (e.g., the abdominal region) or the eye region of the human. In certain embodiments, the formulation is administered or provided to the human subcutaneously as a peri-orbital or intra-orbital injection. In some embodiments, the sterile reconstituted lyophile fluticasone formulation is administered or provided to the human subcutaneously to an abdominal region or an ophthalmic region of a human.

Methods of Manufacturing Fluticasone Lyophile Compositions

In a fifth aspect, provided herein are methods of preparing a lyophilized sterile fluticasone composition comprising: (i) solubilizing fluticasone or a pharmaceutically acceptable salt thereof, a bulking agent, and a non-ionic surfactant with a solvent or co-solvent to form a bulk solution; (ii) sterilizing the bulk solution; and (iii) lyophilizing the sterilized bulk solution to provide a lyophilized sterile fluticasone composition. In some embodiments, the fluticasone or pharmaceutically acceptable salt thereof that is solubilized further comprises a buffering agent that is citric acid or sodium citrate dihydrate. In one embodiment, the buffering agent is citric acid. In another embodiment, the buffering agent is citrate dihydrate.

In some embodiments, the solvent or co-solvent is selected from tert-butyl alcohol, n-butanol, ethanol, iso-propyl alcohol, dimethyl sulfone, chlorobutanol, or mixtures thereof. In one embodiment, the solvent or co-solvent is tert-butyl alcohol. In another embodiment the method provides for sterilizing the bulk solution comprising passing the bulk solution through a filter. In another embodiment, the filter is a 0.2 micron filter. In yet another embodiment, the method comprises the use of a propionate or a furoate salt of fluticasone. In one embodiment the method comprises the use of a propionate salt.

Also provided herein, in further or additional embodiments, is a method of preparing a lyophilized sterile fluticasone formulation as described herein wherein the non-ionic surfactant that is solubilized is selected from (N,N-Bis[3-(D-gluconamido)propyl]cholamide); Bis(polyethylene glycol bis[imidazoyl carbonyl]); Polyoxyethyleneglycol dodecyl ether; Polyoxyethylenglyceroltriricinoleate 35; decaethylene glycol monododecyl ether; N-decanoyl-N-methylglucamine; n-decyl α-D-glucopyranoside; decyl β-D-maltopyranoside; n-dodecanoyl-N-methylglucamide; n-dodecyl α-D-maltoside; n-dodecyl β-D-maltoside; n-hexadecyl β-D-maltoside; heptaethylene glycol monodecyl ether; heptaethylene glycol monododecyl ether; heptaethylene glycol monotetradecyl ether; hexaethylene glycol monododecyl ether; hexaethylene glycol monohexadecyl ether; hexaethylene glycol monooctadecyl ether; hexaethylene glycol monotetradecyl ether; octylphenyl-polyethylene glycol; methyl-6-O—(N-heptylcarbamoyl)-α-D-glucopyranoside; nonaethylene glycol monododecyl ether; N-nonanoyl-N-methylglucamine; N-nonanoyl-N-methylglucamine; octaethylene glycol monodecyl ether; octaethylene glycol monododecyl ether; octaethylene glycol monohexadecyl ether; octaethylene glycol monooctadecyl ether; octaethylene glycol monotetradecyl ether; octyl-β-D-glucopyranoside; pentaethylene glycol monodecyl ether; pentaethylene glycol monododecyl ether; pentaethylene glycol monohexadecyl ether; pentaethylene glycol monohexyl ether; pentaethylene glycol monooctadecyl ether; pentaethylene glycol monooctyl ether; polyethylene glycol diglycidyl ether; polyethylene glycol ether W-1; polyoxyethylene 10 tridecyl ether; polyoxyethylene 100 stearate; polyoxyethylene 20 isohexadecyl ether; polyoxyethylene 20 oleyl ether; polyoxyethylene 40 stearate; polyoxyethylene 50 stearate; polyoxyethylene 8 stearate, polyoxyethylene bis(imidazolyl carbonyl), polyoxyethylene 25 propylene glycol stearate; saponin; sorbitan laurate, sorbitan monopalmitate, sorbitan stearate, sorbitan tristearate, sorbitan oleate, sorbitan trioleate; polyethyelene glycol trimethylnonyl ether, nonylphenol ethoxylate; tetradecyl-β-D-maltoside; tetraethylene glycol monodecyl ether, tetraethylene glycol monododecyl ether, tetraethylene glycol monotetradecyl ether; triethylene glycol monodecyl ether, triethylene glycol monododecyl ether, triethylene glycol monohexadecyl ether, triethylene glycol monooctyl ether, triethylene glycol monotetradecyl ether; octoxynol-9; octylphenol ethoxylate; polysorbate 20, polysorbate 21, polysorbate 40, polysorbate 60, polysorbate 61, polysorbate 65, polysorbate 80, polysorbate 81, polysorbate 85; tyloxapol; and n-undecyl β-D-glucopyranoside. In a specific embodiment, the lyophile fluticasone composition comprises a non-ionic surfactant that is polysorbate 80.

In another embodiment, the bulking agent is selected from the group consisting of lactose, mannitol, dextrose, and sucrose. In yet another embodiment the bulking agent is lactose monohydrate. In yet another embodiment the method provides a bulk solution having a glass transition temperature of about −35° C. to about −25° C.

Methods of Manufacturing Reconstituted Fluticasone Formulations

In a sixth aspect, provided herein are methods for preparing by reconstitution a sterile fluticasone formulation that is suitable for subcutaneous injection comprising the step of contacting a lyophilized sterile fluticasone composition that further comprises a bulking agent and a non-ionic surfactant with a pharmaceutically acceptable diluent or carrier. In some embodiments, the sterile fluticasone lyophile further comprises a buffering agent. In some embodiments, the buffering agent is citric acid or sodium citrate dihydrate. In one embodiment, the buffering agent is citric acid. In another embodiment, the buffering agent is citrate dihydrate. In some embodiments, the pharmaceutically acceptable diluent or carrier is selected from Sterile Water for Injection, 0.9% sodium chloride solution, or 5% dextrose solution. In one embodiment, a pharmaceutically acceptable diluent or carrier that is Sterile Water for Injection is contacted with a fluticasone lyophile to provide a reconstituted sterile fluticasone formulation. In further or additional embodiments, the non-ionic surfactant of the sterile fluticasone lyophile composition that is reconstituted is present in the lyophile fluticasone composition in an amount of about 4% to about 5% of the total combined dry weight of the lyophile.

In some embodiments, provided here is a method of reconstituting a sterile lyophilized fluticasone composition that provides a solubilized or dissolved amount of fluticasone that is at least about 50% of the amount of the lyophilized fluticasone present prior to reconstitution as determined by HPLC after about 2 minutes of dissolution. In some embodiments, provided herein are reconstitution methods of a sterile lyophilized fluticasone composition that provides a solubilized or dissolved amount of fluticasone that is at least about 60% of the amount of lyophilized fluticasone present prior to reconstitution as determined by HPLC after about 2 minutes of dissolution. In yet further or additional embodiments, the methods of reconstituting a sterile lyophilized fluticasone composition described herein provides a solubilized or dissolved amount of fluticasone that is at least about 75% of the amount of lyophilized fluticasone present prior to reconstitution as determined by HPLC after about 2 minutes of dissolution. In yet further or additional embodiments, the methods described herein reconstitute a sterile lyophilized fluticasone composition and provided is a solubilized or dissolved amount of fluticasone that is about 90% to about 100% of the amount of lyophilized fluticasone present prior to reconstitution as determined by HPLC after about 2 minutes of dissolution. In still further or additional embodiments, the reconstitution methods of a sterile lyophilized fluticasone composition provided herein reconstitute a sterile fluticasone composition wherein the non-ionic surfactant was present in the lyophile fluticasone composition prior to reconstitution in an amount of about 4.5% to about 5% of the total combined dry weight of the lyophile. In still further or additional embodiments, the reconstitution methods of the lyophilized fluticasone compositions described herein further comprise the step of adding salmeterol or a pharmaceutically acceptable salt thereof to the reconstituted formulation.

### Lyophilized Salmeterol Compositions

In a seventh aspect, provided herein is a sterile salmeterol lyophile composition comprising lyophilized salmeterol or a pharmaceutically acceptable salt thereof and a bulking agent. In some embodiments, the sterile salmeterol lyophile further comprises a buffering agent. In some embodiments, the buffering agent is citric acid or sodium citrate dihydrate. In one embodiment, the buffering agent is citric acid. In another embodiment, the buffering agent is citrate dihydrate. In still further or additional embodiments, the sterile salmeterol lyophile further comprises a non-ionic surfactant. In one embodiment the lyophile salmeterol composition further comprises an anti-oxidant.

Also described herein is a lyophile composition comprising lyophilized salmeterol in a crystalline phase, an amorphous phase, a semi-crystalline phase, a semi-amorphous phase, or a mixture thereof. In one embodiment, the lyophilized salmeterol is in an amorphous phase. In one embodiment, the pharmaceutically acceptable salt of salmeterol is the xinafoate salt. In one embodiment, the lyophilized salmeterol xinafoate is in a crystalline phase, a polymorph form, an amorphous phase, a semi-crystalline phase, a semi-amorphous phase, or a mixture thereof. In another embodiment, the polymorph form is a Form I polymorph. In a further embodiment, the polymorph form is a Form II polymorph. In yet a further embodiment, the lyophilized salmeterol xinafoate or a pharmaceutically acceptable salt thereof is in an amorphous form.

In some embodiments, provided herein is a sterile salmeterol lyophile composition comprising lyophilized salmeterol or a pharmaceutically acceptable salt thereof and a bulking agent wherein the lyophilized salmeterol is present in an amount of about 0.00001% to about 0.1% by weight of the total combined dry weight of the lyophile. In some embodiments, the sterile lyophilized salmeterol is present in an amount of about 0.0002% to about 0.001% by weight of the total combined dry weight of the lyophile. In further or additional embodiments, the sterile lyophilized salmeterol is present in an amount of about 0.00034% to about 0.00046% by weight of the total combined dry weight of the lyophile. In some embodiments the salmeterol is salmeterol xinafoate.

Also provided herein is a sterile salmeterol lyophile composition comprising lyophilized salmeterol or a pharmaceutically acceptable salt thereof and a bulking agent wherein the sterile lyophilized salmeterol is present in an amount that is equal to or less than about 250 micrograms/gram. In some embodiments, further provided herein is a sterile salmeterol lyophile composition wherein the lyophilized salmeterol is present in an amount of about 100 nanograms/gram to about 250 micrograms/gram. In certain embodiments, provided herein is a sterile salmeterol lyophile composition wherein the lyophilized salmeterol is present in an amount of about 250 nanograms/gram to about 225 micrograms/gram. In some embodiments, provided is a sterile salmeterol lyophile composition comprising lyophilized salmeterol that is present in an amount of about 500 nanograms/gram to about 200 micrograms/gram. In further or additional embodiments, provided is a sterile salmeterol lyophile composition wherein the lyophilized salmeterol is present in an amount of about 1 microgram/gram to about 175 micrograms/gram. On some embodiments, provided is a sterile salmeterol lyophile composition wherein the lyophilized salmeterol is present in an amount of about 2 micrograms/gram to about 150 micrograms/gram. In certain embodiments, provided is a sterile salmeterol lyophile composition that comprises lyophilized salmeterol that is present in an amount of about 5 micrograms/gram to about 125 micrograms/gram. In yet further or additional embodiments, provided is a sterile salmeterol lyophile composition that comprises lyophilized salmeterol that is present in an amount of about 25 micrograms/gram to about 115 micrograms/gram. In some embodiments, provided is a sterile salmeterol lyophile composition wherein the lyophilized salmeterol is present in an amount of about 50 micrograms/gram to about 100 micrograms/gram. In one embodiment, provided is a sterile salmeterol lyophile composition that comprises lyophilized salmeterol that is present in an amount of about 60 micrograms/gram to about 80 micrograms/gram.

Also provided herein is a sterile salmeterol lyophile composition comprising lyophilized salmeterol or a pharmaceutically acceptable salt thereof and a bulking agent wherein the sterile lyophilized salmeterol is present in an amount that is equal to or less than about 500 nanograms/gram. In certain embodiments, the sterile salmeterol lyophile composition comprises sterile lyophilized salmeterol that is present in an amount of about 25 nanograms/gram to about 500 nanograms/gram. In further or additional embodiments, the sterile salmeterol lyophile composition comprises sterile lyophilized salmeterol that is present in an amount of about 100 nanograms/gram to about 350 nanograms/gram. In one embodiment, the sterile salmeterol lyophile composition comprises sterile lyophilized salmeterol that is present in an amount of about 200 nanograms/gram to about 250 nanograms/gram.

In some embodiments, provided herein is a sterile salmeterol lyophile composition comprising a non-ionic surfactant that is selected from (N,N-Bis[3-(D-gluconamido)propyl]cholamide); Bis(polyethylene glycol bis[imidazoyl carbonyl]); Polyoxyethyleneglycol dodecyl ether; Polyoxyethylenglyceroltriricinoleate 35; decaethylene glycol monododecyl ether; N-decanoyl-N-methylglucamine; n-decyl α-D-glucopyranoside; decyl β-D-maltopyranoside; n-dodecanoyl-N-methylglucamide; n-dodecyl α-D-maltoside; n-dodecyl β-D-maltoside; n-hexadecyl β-D-maltoside; heptaethylene glycol monodecyl ether; heptaethylene glycol monododecyl ether; heptaethylene glycol monotetradecyl ether; hexaethylene glycol monododecyl ether; hexaethylene glycol monohexadecyl ether; hexaethylene glycol monooctadecyl ether; hexaethylene glycol monotetradecyl ether; octylphenyl-polyethylene glycol; methyl-6-O-(N-heptylcarbamoyl)-α-D-glucopyranoside; nonaethylene glycol monododecyl ether; N-nonanoyl-N-methylglucamine; N-nonanoyl-N-methylglucamine; octaethylene glycol monodecyl ether; octaethylene glycol monododecyl ether; octaethylene glycol monohexadecyl ether; octaethylene glycol monooctadecyl ether; octaethylene glycol monotetradecyl ether; octyl-β-D-glucopyranoside; pentaethylene glycol monodecyl ether; pentaethylene glycol monododecyl ether; pentaethylene glycol monohexadecyl ether; pentaethylene glycol monohexyl ether; pentaethylene glycol monooctadecyl ether; pentaethylene glycol monooctyl ether; polyethylene glycol diglycidyl ether; polyethylene glycol ether W-1; polyoxyethylene 10 tridecyl ether; polyoxyethylene 100 stearate; polyoxyethylene 20 isohexadecyl ether; polyoxyethylene 20 oleyl ether; polyoxyethylene 40 stearate; polyoxyethylene 50 stearate; polyoxyethylene 8 stearate, polyoxyethylene bis(imidazolyl carbonyl), polyoxyethylene 25 propylene glycol stearate; saponin; sorbitan laurate, sorbitan monopalmitate, sorbitan stearate, sorbitan tristearate, sorbitan oleate, sorbitan trioleate; polyethyelene glycol trimethylnonyl ether, nonylphenol ethoxylate; tetradecyl-β-D-maltoside; tetraethylene glycol monodecyl ether, tetraethylene glycol monododecyl ether, tetraethylene glycol monotetradecyl ether; triethylene glycol monodecyl ether, triethylene glycol monododecyl ether, triethylene glycol monohexadecyl ether, triethylene glycol monooctyl ether, triethylene glycol monotetradecyl ether; octoxynol-9; octylphenol ethoxylate; polysorbate 20, polysorbate 21, polysorbate 40, polysorbate 60, polysorbate 61, polysorbate 65, polysorbate 80, polysorbate 81, polysorbate 85; tyloxapol; and n-undecyl β-D-glucopyranoside. In one embodiment, the sterile lyophile salmeterol composition comprises a non-ionic surfactant that is polysorbate 80.

In some embodiments, provided herein is a sterile salmeterol lyophile composition comprising lyophilized salmeterol or a pharmaceutically acceptable salt thereof and a bulking agent wherein the bulking agent is selected from the group consisting of lactose, mannitol, dextrose, and sucrose. In certain embodiments, the bulking agent is lactose monohydrate.

In further or additional embodiments, provided herein is a sterile salmeterol lyophile composition comprising lyophilized salmeterol or a pharmaceutically acceptable salt thereof, a bulking agent and a non-ionic surfactant wherein the non-ionic surfactant is present in an amount of about 1% to about 10% by weight of the total combined dry weight of the lyophile and/or the bulking agent is present in an amount of about 90% to about 99.9% by weight of the total combined dry weight of the lyophile. In yet further or additional embodiments, the non-ionic surfactant is present in an amount of about 2.5% to about 7.5% by weight or the bulking agent is present in an amount of about 93% to about 97% by weight of the total combined dry weight of the lyophile. In still further embodiments, the non-ionic surfactant is present in an amount of about 4.2% to about 5.8% by weight or the bulking agent is present in an amount of about 95% by weight of the total combined dry weight of the lyophile.

In some embodiments, provided herein is a sterile salmeterol lyophile composition comprising lyophilized salmeterol or a pharmaceutically acceptable salt thereof, a bulking agent, and an anti-oxidant wherein the anti-oxidant is ascorbic acid or butylated hydroxytoluene. In certain embodiments, the composition is stable for a period of at least 4 weeks at a temperature of about 0° C. to about 50° C. In still further or additional embodiments, the composition is stable for a period of at least 6 months at a temperature of about 0° C. to about 50° C. In some embodiments, the sterile salmeterol lyophile composition further comprises a buffering agent. In some embodiments, the buffering agent is citric acid or sodium citrate dihydrate. In one embodiment, the buffering agent is citric acid. In another embodiment, the buffering agent is citrate dihydrate. In still further or additional embodiments, the sterile salmeterol lyophile further comprises a non-ionic surfactant. In one embodiment the lyophile salmeterol composition further comprises an anti-oxidant.

Also provided herein are sterile salmeterol lyophile compositions comprising lyophilized salmeterol or a pharmaceutically acceptable salt thereof and a bulking agent wherein the composition is suitable, upon reconstitution with an aqueous solution, for subcutaneous peri-orbital or intra-orbital. In certain embodiments, the composition is suitable, upon reconstitution with an aqueous solution, for subcutaneous administration. In some embodiments, the composition contains less than about 3% solvent or co-solvent by weight of the total combined dry weight of the lyophile. In further or additional embodiments, the composition contains less than about 2% solvent or co-solvent by weight of the total combined dry weight of the lyophile. In still further embodiments, the composition contains less than about 1% solvent or co-solvent by weight of the total combined dry weight of the lyophile. In some embodiments, the solvent or co-solvent is selected from tert-butyl alcohol, n-butanol, ethanol, iso-propyl alcohol, dimethyl sulfone, chlorobutanol, Sterile Water for Injection, 0.9% sodium chloride solution, 5% dextrose solution, or mixtures thereof. In still further embodiments, the solvent or co-solvent is tert-butyl alcohol or ethanol. In one embodiment, the co-solvent is ethanol. In another embodiment, the composition contains less than about 2% water by weight of the total combined dry weight of the lyophile.

Reconstituted Salmeterol Formulations

In an eighth aspect, provided herein are sterile salmeterol formulations that have been reconstituted from a lyophilized composition that comprised lyophilized salmeterol or a pharmaceutically acceptable salt thereof and a bulking agent. In further or additional embodiments, the sterile salmeterol formulation has been reconstituted from a lyophilized composition that further comprised a non-ionic surfactant, an anti-oxidant, and/or a buffer. In some embodiments, the sterile salmeterol formulation has been reconstituted from a lyophilized composition that further comprised a non-ionic surfactant. In one embodiment, the sterile salmeterol formulation has been reconstituted from a lyophilized composition that further comprised an anti-oxidant. In another embodiment, the sterile salmeterol lyophile composition further comprised a buffering agent. In some embodiments, the buffering agent is citric acid or sodium citrate dihydrate. In one embodiment, the buffering agent is citric acid. In another embodiment, the buffering agent is citrate dihydrate.

In some embodiments, the reconstituted sterile salmeterol formulation further comprises a pharmaceutically acceptable diluent that is selected from Sterile Water for Injection, 0.9% sodium chloride solution, or 5% dextrose solution. In certain embodiments, the pharmaceutically acceptable diluent is Sterile Water for Injection.

In some embodiments, the reconstituted sterile salmeterol formulation comprises salmeterol that is present in an amount that is equal to or less than about 25 micrograms/milliliter. In further or additional embodiments, the reconstituted sterile salmeterol formulation comprises salmeterol that is present in an amount that is equal to or less than about 1 microgram/milliliter. In further or additional embodiments, the reconstituted sterile salmeterol formulation comprises salmeterol that is present in an amount of about 2 nanograms/milliliter to about 25 micrograms/milliliter. In yet further or additional embodiments, the reconstituted sterile salmeterol formulation comprises salmeterol that is present in an amount of about 2 nanograms/milliliter to about 1 microgram/milliliter.

In yet further or additional embodiments, the reconstituted sterile salmeterol formulation comprises salmeterol that is present in an amount of about 4 nanograms/milliliter to about 750 nanograms/milliliter. In some embodiments, the reconstituted sterile salmeterol formulation comprises salmeterol that is present in an amount of about 5 nanogram/milliliter to about 500 nanograms/milliliter. In still further or additional embodiments, the reconstituted sterile salmeterol formulation comprises salmeterol that is present in an amount of about 10 nanograms/milliliter to about 250 nanograms/milliliter. In some embodiments, the reconstituted sterile salmeterol formulation comprises salmeterol that is present in an amount of about 15 nanograms/milliliter to about 50 nanograms/milliliter. In yet additional embodiments, the reconstituted sterile salmeterol formulation comprises salmeterol that is present in an amount of about 20 nanograms/milliliter to about 25 nanograms/milliliter. In one embodiment, the reconstituted sterile salmeterol formulation comprises salmeterol that is present in an amount of about 20 nanograms/milliliter.

In certain embodiments, the reconstituted sterile salmeterol formulation is formulated for subcutaneous, including peri-orbital or intra-orbital, injection. In additional embodiments, the reconstituted sterile salmeterol formulation is formulated for subcutaneous administration. In some embodiments, the reconstituted sterile salmeterol formulation is dissolved in the pharmaceutically acceptable diluent. In still further embodiments, the dissolution occurs in less than about 2 minutes. In some embodiments, the dissolution occurs in about 1 minute. In still further embodiments, the dissolution occurs in less than about 1 minute. In yet further or additional embodiments, the dissolution results in a clear solution.

Reconstituted Salmeterol Formulation Further Comprising Fluticasone

In a ninth aspect, provided herein are sterile salmeterol formulations that have been reconstituted from a lyophilized composition that comprised lyophilized salmeterol or a pharmaceutically acceptable salt thereof and a bulking agent. In further or additional embodiments, the sterile salmeterol formulation has been reconstituted from a lyophilized composition that further comprised a non-ionic surfactant, an anti-oxidant, and/or a buffer. In some embodiments, the sterile salmeterol formulation has been reconstituted from a lyophilized composition that further comprised a non-ionic surfactant. In one embodiment, the sterile salmeterol formulation has been reconstituted from a lyophilized composition that further comprised an anti-oxidant. In another embodiment, the sterile salmeterol lyophile composition further comprised a buffering agent. In some embodiments, the buffering agent is citric acid or sodium citrate dihydrate. In one embodiment, the buffering agent is citric acid. In another embodiment, the buffering agent is citrate dihydrate.

In some embodiments, the reconstituted sterile salmeterol formulation comprises salmeterol and is further formulated to comprise fluticasone or a pharmaceutically acceptable salt thereof. In some embodiments, the fluticasone has been reconstituted from a sterile lyophile composition. In other embodiments, the fluticasone has not been reconstituted from a sterile lyophile composition. In some embodiments, the reconstituted sterile salmeterol formulation comprising salmeterol and fluticasone further comprises a pharmaceutically acceptable diluent that is selected from Sterile Water for Injection, 0.9% sodium chloride solution, or 5% dextrose solution. In certain embodiments, the pharmaceutically acceptable diluent is Sterile Water for Injection.

In some embodiments, provided is a sterile salmeterol formulation that has been reconstituted from a lyophilized composition that comprised lyophilized salmeterol or a pharmaceutically acceptable salt thereof, a bulking agent, a non-ionic surfactant, and an anti-oxidant, wherein the formulation further comprises fluticasone or a pharmaceutically acceptable salt thereof that is present in an amount of about 100 nanograms/milliliter to about 100 micrograms/milliliter. In further or additional embodiments, the fluticasone is present in an amount of about 200 nanograms/milliliter to about 50 micrograms/milliliter. In some embodiments, the fluticasone is present in an amount of about 300 nanograms/milliliter to about 25 micrograms/milliliter. In yet additional embodiments, the fluticasone is present in an amount of about 500 nanograms/milliliter to about 15 micrograms/milliliter. In certain embodiments, the fluticasone is present in an amount of about 600 nanograms/milliliter to about 10 micrograms/milliliter. In still further or additional embodiments, the fluticasone is present in an amount of about 750 nanograms/milliliter to about 5 micrograms/milliliter. In some embodiments, the fluticasone is present in an amount of about 800 nanograms/milliliter to about 3 micrograms/milliliter. In still further embodiments, the fluticasone is present in an amount of about 900 nanograms/milliliter to about 2 micrograms/milliliter. In one embodiment, the fluticasone is present in an amount of about 1 microgram/milliliter. In some embodiments, the reconstituted sterile formulation comprising salmeterol and fluticasone is formulated for subcutaneous peri-orbital or intra-orbital injection. In certain embodiments, the reconstituted sterile formulation is formulated for subcutaneous administration by injection.

Therapeutic and Cosmetic Methods of Using Salmeterol

In a tenth aspect, provided herein are cosmetic or therapeutic methods comprising administering or providing, including for example by subcutaneous administration, to a human a sterile salmeterol formulation that has been reconstituted from a lyophilized composition that comprised lyophilized salmeterol or a pharmaceutically acceptable salt thereof and a bulking agent. In some embodiments, the lyophilized salmeterol composition further comprised a non-ionic surfactant, an anti-oxidant, and/or a buffering agent. In some embodiments, the buffering agent is citric acid or sodium citrate dihydrate. In one embodiment, the buffering agent is citric acid. In another embodiment, the buffering agent is citrate dihydrate. In some embodiments, the method is therapeutic. In further or additional embodiments, the method is cosmetic.

In some embodiments, the reconstituted sterile salmeterol formulation is used (for example by provision or administration) to treat an indication selected from the group consisting of abdominal adiposity, regional adiposity, and exophthalmos due to thyroid eye disease. In certain embodiments, the reconstituted sterile salmeterol formulation is administered or provided subcutaneously. In some embodiments, the reconstituted sterile salmeterol formulation is administered or provided to the human subcutaneously as a peri-orbital or intra-orbital injection. In some embodiments, the reconstituted sterile salmeterol formulation is administered or provided to the human subcutaneously to an abdominal region or an ophthalmic region. In further or additional embodiments, provided is a reconstituted salmeterol formulation wherein the formulation is administered or provided to the human in the inside region of the knees, the middle to upper area of the upper arm (including the tricep area), the submental area (including the area under the chin, for example the wattle (which is understood to refer to the fleshy fold of skin in the submental area of a patient)), the abdomen, the hips, the inner thigh, the outer thigh, the buttocks, the lower back, the upper back or the chest.

In further embodiments, provided herein is a cosmetic method comprising administering or providing, including for example by subcutaneous administration, to a human a sterile salmeterol formulation that has been reconstituted from a lyophilized composition that comprised lyophilized salmeterol or a pharmaceutically acceptable salt thereof and a bulking agent. In some embodiments, the lyophilized salmeterol composition further comprised a non-ionic surfactant, an anti-oxidant, and/or a buffering agent. In some embodiments, the buffering agent is citric acid or sodium citrate dihydrate. In one embodiment, the buffering agent is citric acid. In another embodiment, the buffering agent is citrate dihydrate. In some embodiments, the method is therapeutic. In further or additional embodiments, the method is cosmetic.

In some embodiments, the reconstituted sterile salmeterol formulation is provided cosmetically to the human to affect a shape, contour, or appearance of the human body. In further or additional embodiments, the shape, contour, or appearance is in a region of the body (e.g., the abdominal region) or the eye region of the human. In certain embodiments, the formulation is administered or provided to the human subcutaneously as a peri-orbital or intra-orbital injection. In some embodiments, the sterile reconstituted salmeterol formulation is administered or provided to the human subcutaneously to an abdominal region or an ophthalmic region of a human.

Methods of Manufacturing Salmeterol Lyophile Compositions

In an eleventh aspect, provided herein are methods of preparing a lyophilized sterile salmeterol composition comprising: (i) solubilizing salmeterol or a pharmaceutically acceptable salt thereof and a bulking agent with a solvent or co-solvent to form a bulk solution; (ii) sterilizing the bulk solution; and (iii) lyophilizing the sterilized bulk solution to provide a lyophilized sterile salmeterol xinafoate composition. In some embodiments, the salmeterol or a pharmaceutically acceptable salt thereof that is solubilized further comprises a non-ionic surfactant, an anti-oxidant, and/or a buffering agent. In some embodiments, the buffering agent is citric acid or sodium citrate dihydrate. In one embodiment, the buffering agent is citric acid. In another embodiment, the buffering agent is citrate dihydrate.

In some embodiments, the solvent or co-solvent is selected from tert-butyl alcohol, n-butanol, ethanol, iso-propyl alcohol, dimethyl sulfone, chlorobutanol, Sterile Water for Injection, 0.9% sodium chloride solution, 5% dextrose solution, or mixtures thereof. In some embodiments, the solvent is tert-butyl alcohol. In one embodiment, the solvent is ethanol. In certain embodiments, the sterilizing the bulk solution comprises passing the bulk solution through a filter. In yet further or additional embodiments, the filter is a 0.2 micron filter. In some embodiments, the salmeterol is salmeterol xinafoate.

In some embodiments, non-ionic surfactant is selected from (N,N-Bis[3-(D-gluconamido)propyl]cholamide); Bis(polyethylene glycol bis[imidazoyl carbonyl]); Polyoxyethyleneglycol dodecyl ether; Polyoxyethylenglyceroltriricinoleate 35; decaethylene glycol monododecyl ether; N-decanoyl-N-methylglucamine; n-decyl α-D-glucopyranoside; decyl β-D-maltopyranoside; n-dodecanoyl-N-methylglucamide; n-dodecyl α-D-maltoside; n-dodecyl β-D-maltoside; n-hexadecyl β-D-maltoside; heptaethylene glycol monodecyl ether; heptaethylene glycol monododecyl ether; heptaethylene glycol monotetradecyl ether; hexaethylene glycol monododecyl ether; hexaethylene glycol monohexadecyl ether; hexaethylene glycol monooctadecyl ether; hexaethylene glycol monotetradecyl ether; octylphenyl-polyethylene glycol; methyl-6-O-(N-heptylcarbamoyl)-α-D-glucopyranoside; nonaethylene glycol monododecyl ether; N-nonanoyl-N-methylglucamine; N-nonanoyl-N-methylglucamine; octaethylene glycol monodecyl ether; octaethylene glycol monododecyl ether; octaethylene glycol monohexadecyl ether; octaethylene glycol monooctadecyl ether; octaethylene glycol monotetradecyl ether; octyl-β-D-glucopyranoside; pentaethylene glycol monodecyl ether; pentaethylene glycol monododecyl ether; pentaethylene glycol monohexadecyl ether; pentaethylene glycol monohexyl ether; pentaethylene glycol monooctadecyl ether; pentaethylene glycol monooctyl ether; polyethylene glycol diglycidyl ether; polyethylene glycol ether W-1; polyoxyethylene 10 tridecyl ether; polyoxyethylene 100 stearate; polyoxyethylene 20 isohexadecyl ether; polyoxyethylene 20 oleyl ether; polyoxyethylene 40 stearate; polyoxyethylene 50 stearate; polyoxyethylene 8 stearate, polyoxyethylene bis(imidazolyl carbonyl), polyoxyethylene 25 propylene glycol stearate; saponin; sorbitan laurate, sorbitan monopalmitate, sorbitan stearate, sorbitan tristearate, sorbitan oleate, sorbitan trioleate; polyethyelene glycol trimethylnonyl ether, nonylphenol ethoxylate; tetradecyl-β-D-maltoside; tetraethylene glycol monodecyl ether, tetraethylene glycol monododecyl ether, tetraethylene glycol monotetradecyl ether; triethylene glycol monodecyl ether, triethylene glycol monododecyl ether, triethylene glycol monohexadecyl ether, triethylene glycol monooctyl ether, triethylene glycol monotetradecyl ether; octoxynol-9; octylphenol ethoxylate; polysorbate 20, polysorbate 21, polysorbate 40, polysorbate 60, polysorbate 61, polysorbate 65, polysorbate 80, polysorbate 81, polysorbate 85; tyloxapol; and n-undecyl β-D-glucopyranoside. In one embodiment, the non-ionic surfactant is polysorbate 80.

In some embodiments of the methods of preparing a lyophilized sterile salmeterol xinafoate composition described herein, the bulking agent used in the method is selected from the group consisting of lactose, mannitol, dextrose, and sucrose. In one embodiment, the bulking agent is lactose monohydrate. In further or additional embodiments, the sterile lyophilized salmeterol composition comprises an anti-oxidant. In one embodiment, the anti-oxidant is ascorbic acid. In another embodiment, the anti-oxidant is butylated hydroxytoluene. In yet further or additional embodiments of the methods of preparing a lyophilized sterile salmeterol composition described herein, the bulk solution has a glass transition temperature of about −35° C. to about −25° C.

Methods of Manufacturing Reconstituted Salmeterol Formulations

In a twelfth aspect, provided herein are methods for preparing by reconstitution a sterile salmeterol formulation that is suitable for subcutaneous injection comprising the step of contacting a lyophilized sterile salmeterol composition that further comprises a bulking agent with a pharmaceutically acceptable diluent or carrier. In some embodiments, the lyophilized sterile composition further comprises a non-ionic surfactant, an anti-oxidant, and/or a buffering agent. In some embodiments, the lyophile further comprises a buffering agent. In further or additional embodiments, the buffering agent is citric acid or sodium citrate dihydrate. In one embodiment, the buffering agent is citric acid. In another embodiment, the buffering agent is citrate dihydrate.

In some embodiments, the pharmaceutically acceptable diluent or carrier is selected from Sterile Water for Injection, 0.9% sodium chloride solution, or 5% dextrose solution. In one embodiment, a pharmaceutically acceptable diluent or carrier that is Sterile Water for Injection is contacted with a salmeterol lyophile to provide a reconstituted sterile salmeterol formulation. In further or additional embodiments, the non-ionic surfactant of the sterile salmeterol lyophile composition that is reconstituted is present in the lyophile salmeterol composition in an amount of about 4% to about 5% of the total combined dry weight of the lyophile.

In further or additional embodiments, the method of preparing by reconstitution a sterile salmeterol formulation that is suitable for subcutaneous injection provides a solubilized or dissolved amount of salmeterol in the formulation that is about 50% to about 100% of the amount of lyophilized salmeterol present in the composition prior to reconstitution as determined by HPLC after about 2 minutes of dissolution. In some embodiments, the method of preparing by reconstitution a sterile salmeterol formulation that is suitable for subcutaneous injection provides a solubilized or dissolved amount of salmeterol in the formulation that is at least about 75% of the amount of lyophilized salmeterol present in the composition prior to reconstitution as determined by HPLC after about 2 minutes of dissolution. In further or additional embodiments, the method of preparing by reconstitution a sterile salmeterol formulation that is suitable for subcutaneous injection provides a solubilized or dissolved amount of salmeterol in the formulation that is about 90% to about 100% of the amount of lyophilized salmeterol present in the composition prior to reconstitution as determined by HPLC after about 2 minutes of dissolution. In yet further or additional embodiments, the method of preparing by reconstitution a sterile salmeterol formulation that is suitable for subcutaneous injection further comprises the step of adding fluticasone or a pharmaceutically acceptable salt thereof to the formulation.

### Lyophilized Fluticasone and Salmeterol Compositions

In a thirteenth aspect, provided herein is a sterile fluticasone and salmeterol lyophile composition comprising: (a) a lyophilized fluticasone or a pharmaceutically acceptable salt thereof; (b) a lyophilized salmeterol or a pharmaceutically acceptable salt thereof; (c) a bulking agent; and (d) a non-ionic surfactant. In some embodiments, the lyophilized composition further comprises an anti-oxidant and/or a buffering agent. In some embodiments, the lyophile further comprises a buffering agent. In further embodiments, the buffering agent is citric acid or sodium citrate dihydrate. In one embodiment, the buffering agent is citric acid. In another embodiment, the buffering agent is citrate dihydrate.

In some embodiments, the salmeterol is the salmeterol xinafoate salt form. In further or additional embodiments, the salmeterol xinafoate is a polymorph. In certain embodiments, the salmeterol polymorph is polymorph Form I. In additional embodiments, the salmeterol polymorph is Form II. In still further or additional embodiments, the salmeterol is in a crystalline phase, an amorphous phase, a semi-crystalline phase, a semi-amorphous phase, or a mixture thereof. In one embodiment, the salmeterol is amorphous.

In some embodiments, the fluticasone and salmeterol lyophile composition comprises fluticasone propionate or fluticasone furoate. In one embodiment, the fluticasone is fluticasone propionate salt form. In some embodiments, the lyophilized fluticasone is in a crystalline phase, an amorphous phase, a semi-crystalline phase, a semi-amorphous phase, or a mixture thereof. In further or additional embodiments, the lyophilized fluticasone or a pharmaceutically acceptable salt thereof is in a crystalline phase, an amorphous phase, a semi-crystalline phase, a semi-amorphous phase, or a mixture thereof; and the lyophilized salmeterol is in a crystalline phase, a polymorph form, an amorphous phase, a semi-crystalline phase, a semi-amorphous phase, or a mixture thereof.

In further or additional embodiments, provided is a sterile fluticasone and salmeterol lyophile composition that further comprises an anti-oxidant. For example, in some embodiments, the anti-oxidant is ascorbic acid or butylated hydroxytoluene. In still further or additional embodiments, provided is a sterile fluticasone and salmeterol lyophile composition that further comprises a buffering agent. In some embodiments, the buffering agent is citric acid or sodium citrate dihydrate. In one embodiment, the buffering agent is citric acid. In another embodiment, the buffering agent is citrate dihydrate.

In some embodiments, provided is a sterile fluticasone and salmeterol lyophile composition wherein the ratio of fluticasone to salmeterol is about 200:1 to about 1:1. In further or additional embodiments, the ratio of fluticasone to salmeterol is about 150:1 to about 1:1. In still further or additional embodiments, the ratio of fluticasone to salmeterol is about 100:1 to about 1:1. In yet further or additional embodiments, the ratio of fluticasone to salmeterol is about 50:1 to about 1:1. In some embodiments, the ratio of fluticasone to salmeterol is about 20:1 to about 1:1. In further or additional embodiments, the ratio of fluticasone to salmeterol is about 10:1 to about 1:1. In yet further or additional embodiments, the ratio of fluticasone to salmeterol is about 50:1.

Also provided herein are sterile fluticasone and salmeterol lyophile compositions comprising: (a) a lyophilized fluticasone or a pharmaceutically acceptable salt thereof; (b) a lyophilized salmeterol or a pharmaceutically acceptable salt thereof; (c) a bulking agent; and (d) a non-ionic surfactant, wherein the lyophilized fluticasone is present in an amount of about 0.001% to about 0.1% by weight of the total combined dry weight of the lyophile, and the salmeterol is present in an amount of about 0.00001% to about 0.1% by weight of the total combined dry weight of the lyophile. In certain embodiments of the sterile fluticasone and salmeterol lyophile compositions described herein, the lyophilized fluticasone is present in an amount of about 0.015% to about 0.030% by weight of the total combined dry weight of the lyophile and the lyophilized salmeterol is present in an amount of about 0.0002% to about 0.001% by weight of the total combined dry weight of the lyophile. In some embodiments, provided is a sterile fluticasone and salmeterol lyophile composition wherein the lyophilized fluticasone is present in an amount of about 0.017% to about 0.023% by weight of the total combined dry weight of the lyophile and the salmeterol is present in an amount of about 0.00034% to about 0.00046% by weight of the total combined dry weight of the lyophile.

Described herein are sterile fluticasone and salmeterol lyophile compositions comprising: (a) a lyophilized fluticasone or a pharmaceutically acceptable salt thereof; (b) a lyophilized salmeterol or a pharmaceutically acceptable salt thereof; (c) a bulking agent; and (d) a non-ionic surfactant, wherein the lyophilized fluticasone is present in an amount that is equal to or less than about 500 micrograms/gram and the lyophilized salmeterol is present in an amount equal to or less than about 250 micrograms/gram. In certain embodiments, the lyophilized fluticasone is present in an amount of about 1 microgram/gram to about 500 micrograms/gram and the lyophilized salmeterol is present in an amount of about 100 nanograms/gram to about 250 micrograms/gram. In yet additional embodiments, the lyophilized fluticasone is present in an amount of about 5 micrograms/gram to about 350 micrograms/gram and the lyophilized salmeterol is present in an amount of about 250 nanograms/gram to about 200 micrograms/gram. In certain embodiments, the lyophilized fluticasone is present in an amount of about 15 micrograms/gram to about 300 micrograms/gram and the lyophilized salmeterol is present in an amount of about 500 nanograms/gram to about 150 micrograms/gram. In some embodiments, the lyophilized fluticasone is present in an amount of about 20 micrograms/gram to about 250 micrograms/gram and the lyophilized salmeterol is present in an amount of about 1 microgram/gram to about 100 micrograms/gram. In yet further embodiments, the lyophilized fluticasone is present in an amount of about 30 micrograms/gram to about 200 micrograms/gram and the lyophilized salmeterol is present in an amount of about 2 micrograms/gram to about 50 micrograms/gram. In still further or additional embodiments, the lyophilized fluticasone is present in an amount of about 100 micrograms/gram to about 300 micrograms/gram and the lyophilized salmeterol is present in an amount of about 3 micrograms/gram to about 25 micrograms/gram. In some embodiments, the lyophilized fluticasone is present in an amount of about 200 micrograms/gram to about 300 micrograms/gram and the lyophilized salmeterol is present in an amount of about 4 micrograms/gram to about 15 micrograms/gram. In some embodiments, the lyophilized fluticasone is present in an amount of about 200 micrograms/gram to about 300 micrograms/gram and the lyophilized salmeterol is present in an amount of about 5 micrograms/gram to about 10 micrograms/gram. In yet further or additional embodiments, the lyophilized fluticasone is present in an amount of about 150 micrograms/gram and the lyophilized salmeterol is present in an amount of about 6 micrograms/gram to about 8 micrograms/gram.

In some embodiments, provided are sterile fluticasone and salmeterol lyophile compositions comprising: (a) a lyophilized fluticasone or a pharmaceutically acceptable salt thereof; (b) a lyophilized salmeterol or a pharmaceutically acceptable salt thereof; (c) a bulking agent; and (d) a non-ionic surfactant, wherein the lyophilized fluticasone is present in an amount that is equal to or less than about 50 micrograms/gram and the lyophilized salmeterol is present in an amount that is equal to or less than about 500 nanograms/gram. In some embodiments, the lyophilized fluticasone is present in an amount of about 1 microgram/gram to about 50 micrograms/gram and the lyophilized salmeterol is present in an amount of about 25 nanograms/gram to about 500 nanograms/gram. In certain embodiments, the lyophilized fluticasone is present in an amount of about 5 micrograms/gram to about 35 micrograms/gram and the lyophilized salmeterol is present in an amount of about 100 nanograms/gram to about 350 nanograms/gram. In one embodiment, the lyophilized fluticasone is present in an amount of about 9 micrograms/gram to about 20 micrograms/gram and the lyophilized salmeterol is present in an amount of about 200 nanograms/gram to about 250 nanograms/gram.

In further or additional embodiments, provided are sterile fluticasone and salmeterol lyophile compositions comprising: (a) a lyophilized fluticasone or a pharmaceutically acceptable salt thereof; (b) a lyophilized salmeterol or a pharmaceutically acceptable salt thereof; (c) a bulking agent; and (d) a non-ionic surfactant, wherein the non-ionic surfactant is selected from (N,N-Bis[3-(D-gluconamido)propyl]cholamide); Bis(polyethylene glycol bis[imidazoyl carbonyl]); Polyoxyethyleneglycol dodecyl ether; Polyoxyethylenglyceroltriricinoleate 35; decaethylene glycol monododecyl ether; N-decanoyl-N-methylglucamine; n-decyl α-D-glucopyranoside; decyl β-D-maltopyranoside; n-dodecanoyl-N-methylglucamide; n-dodecyl α-D-maltoside; n-dodecyl β-D-maltoside; n-hexadecyl β-D-maltoside; heptaethylene glycol monodecyl ether; heptaethylene glycol monododecyl ether; heptaethylene glycol monotetradecyl ether; hexaethylene glycol monododecyl ether; hexaethylene glycol monohexadecyl ether; hexaethylene glycol monooctadecyl ether; hexaethylene glycol monotetradecyl ether; octylphenyl-polyethylene glycol; methyl-6-O-(N-heptylcarbamoyl)-α-D-glucopyranoside; nonaethylene glycol monododecyl ether; N-nonanoyl-N-methylglucamine; N-nonanoyl-N-methylglucamine; octaethylene glycol monodecyl ether; octaethylene glycol monododecyl ether; octaethylene glycol monohexadecyl ether; octaethylene glycol monooctadecyl ether; octaethylene glycol monotetradecyl ether; octyl-β-D-glucopyranoside; pentaethylene glycol monodecyl ether; pentaethylene glycol monododecyl ether; pentaethylene glycol monohexadecyl ether; pentaethylene glycol monohexyl ether; pentaethylene glycol monooctadecyl ether; pentaethylene glycol monooctyl ether; polyethylene glycol diglycidyl ether; polyethylene glycol ether W-1; polyoxyethylene 10 tridecyl ether; polyoxyethylene 100 stearate; polyoxyethylene 20 isohexadecyl ether; polyoxyethylene 20 oleyl ether; polyoxyethylene 40 stearate; polyoxyethylene 50 stearate; polyoxyethylene 8 stearate, polyoxyethylene bis(imidazolyl carbonyl), polyoxyethylene 25 propylene glycol stearate; saponin; sorbitan laurate, sorbitan monopalmitate, sorbitan stearate, sorbitan tristearate, sorbitan oleate, sorbitan trioleate; polyethyelene glycol trimethylnonyl ether, nonylphenol ethoxylate; tetradecyl-β-D-maltoside; tetraethylene glycol monodecyl ether, tetraethylene glycol monododecyl ether, tetraethylene glycol monotetradecyl ether; triethylene glycol monodecyl ether, triethylene glycol monododecyl ether, triethylene glycol monohexadecyl ether, triethylene glycol monooctyl ether, triethylene glycol monotetradecyl ether; octoxynol-9; octylphenol ethoxylate; polysorbate 20, polysorbate 21, polysorbate 40, polysorbate 60, polysorbate 61, polysorbate 65, polysorbate 80, polysorbate 81, polysorbate 85; tyloxapol; and n-undecyl β-D-glucopyranoside. In one embodiment, the non-ionic surfactant is polysorbate 80. In still further or additional embodiments, the bulking agent is selected from the group consisting of lactose, mannitol, dextrose, and sucrose. In one embodiment, the bulking agent is lactose. In some embodiments, the bulking agent is lactose monohydrate.

In some embodiments of the sterile fluticasone and salmeterol lyophile compositions, the non-ionic surfactant is present from about 1.0% to about 10% by weight of the total combined dry weight of the lyophile or the bulking agent is present in an amount of about 90% to about 99% by weight of the total combined dry weight of the lyophile. In further or additional embodiments, the non-ionic surfactant is present from about 2.5% to about 7.5% by weight of the total combined dry weight of the lyophile or the bulking agent is present in an amount of about 93% to about 97% by weight of the total combined dry weight of the lyophile. In yet additional embodiments, the non-ionic surfactant is present from about 4.2% to about 5.8% by weight of the total combined dry weight of the lyophile or the bulking agent is present in an amount of about 95% by weight of the total combined dry weight of the lyophile.

In further or additional embodiments of the sterile fluticasone and salmeterol lyophile compositions provided herein, the composition is suitable, upon reconstitution with an aqueous solution, for subcutaneous, peri-orbital, intra-orbital, or intramuscular administration. In some embodiments, the composition is suitable, upon reconstitution with an aqueous solution, for subcutaneous administration. In yet further or additional embodiments, the composition contains less than about 3% solvent or co-solvent by weight of the total combined dry weight of the lyophile. In certain embodiments, the composition contains less than about 2% solvent or co-solvent by weight of the total combined dry weight of the lyophile. In some embodiments, the composition contains less than about 1% solvent or co-solvent by weight of the total combined dry weight of the lyophile. In still further or additional embodiments, the composition contains less than about 2% water by weight of the total combined dry weight of the lyophile. In certain embodiments, the composition is stable for a period of at least 4 weeks at a temperature of about 0° C. to about 50° C. In yet further or additional embodiments, the composition is stable for a period of at least 6 months at a temperature of about 0° C. to about 50° C.

Reconstituted Fluticasone and Salmeterol Formulations

In a fourteenth aspect, provided herein is a sterile formulation that has been reconstituted from a lyophilized composition that comprised fluticasone or a pharmaceutically acceptable salt thereof; salmeterol or a pharmaceutically acceptable salt thereof; a bulking agent; and a non-ionic surfactant. In some embodiments, the lyophilized composition further comprised an anti-oxidant. In some embodiments, the lyophilized sterile composition further comprised an anti-oxidant and/or a buffering agent. In some embodiments, the lyophile further comprised a buffering agent. In further or additional embodiments, the buffering agent is citric acid or sodium citrate dihydrate. In one embodiment, the buffering agent is citric acid. In another embodiment, the buffering agent is citrate dihydrate.

In some embodiments, the reconstituted sterile formulation further comprised a pharmaceutically acceptable diluent that is selected from Sterile Water for Injection, 0.9% sodium chloride solution, or 5% dextrose solution. In one embodiment, the pharmaceutically acceptable diluent is Sterile Water for Injection.

In some embodiments, the reconstituted sterile formulation comprises reconstituted fluticasone that is present in an amount equal to or less than about 100 micrograms/milliliter and reconstituted salmeterol that is present in an amount equal to or less than about 25 micrograms/milliliter. In some embodiments, the reconstituted fluticasone is present in an amount of about 100 nanograms/milliliter to about 100 micrograms/milliliter and the reconstituted salmeterol is present in an amount of about 2 nanograms/milliliter to about 25 micrograms/milliliter. In further or additional embodiments, the reconstituted fluticasone is present in an amount of about 200 nanograms/milliliter to about 50 micrograms/milliliter and the reconstituted salmeterol is present in an amount of about 2 nanograms/milliliter to about 1 microgram/milliliter. In yet further or additional embodiments, the reconstituted fluticasone is present in an amount of about 300 nanograms/milliliter to about 25 micrograms/milliliter and the reconstituted salmeterol is present in an amount of about 2 nanograms/milliliter to about 1 microgram/milliliter. In certain embodiments, the reconstituted fluticasone is present in an amount of about 400 nanograms/milliliter to about 20 micrograms/milliliter and the reconstituted salmeterol is present in an amount of about 4 nanograms/milliliter to about 750 nanograms/milliliter. In still further embodiments, the reconstituted fluticasone is present in an amount of about 500 nanograms/milliliter to about 15 micrograms/milliliter and the reconstituted salmeterol is present in an amount of about 5 nanogram/milliliter to about 500 nanograms/milliliter. In some embodiments, the reconstituted fluticasone is present in an amount of about 600 nanograms/milliliter to about 10 micrograms/milliliter and the reconstituted salmeterol is present in an amount of about 10 nanograms/milliliter to about 250 nanograms/milliliter. In some embodiments, the reconstituted fluticasone is present in an amount of about 750 nanograms/milliliter to about 5 micrograms/milliliter and the reconstituted salmeterol is present in an amount of about 15 nanograms/milliliter to about 50 nanograms/milliliter. In yet further or additional embodiments, the reconstituted fluticasone is present in an amount of about 800 nanograms/milliliter to about 3 micrograms/milliliter and the reconstituted salmeterol is present in an amount of about 20 nanograms/milliliter to about 25 nanograms/milliliter. In some embodiments, the reconstituted fluticasone is present in an amount of about 900 nanograms/milliliter to about 2 micrograms/milliliter the reconstituted salmeterol is present in an amount of about 20 nanograms/milliliter to about 25 nanograms/milliliter. In one embodiment, the reconstituted fluticasone is present in an amount of about 1 microgram/milliliter and the reconstituted salmeterol is present in an amount of about 20 nanograms/milliliter.

In some embodiments, provided herein is a sterile formulation that has been reconstituted from a lyophilized composition that comprised fluticasone or a pharmaceutically acceptable salt thereof; salmeterol or a pharmaceutically acceptable salt thereof; a bulking agent; and a non-ionic surfactant, wherein the formulation provides a solubilized or dissolved amount of fluticasone that is at least about 50% of the amount of the lyophilized fluticasone present prior to reconstitution and/or a solubilized or dissolved amount of salmeterol that is at least about 50% of the amount of the lyophilized salmeterol present prior to reconstitution as determined by HPLC after about 2 minutes of dissolution. In some embodiments, the sterile reconstituted formulation provides a solubilized or dissolved amount of fluticasone that is at least about 60% of the amount of lyophilized fluticasone present prior to reconstitution and/or a solubilized or dissolved amount of salmeterol that is at least about 60% of the amount of the lyophilized salmeterol present prior to reconstitution as determined by HPLC after about 2 minutes of dissolution. In further or additional embodiments, the sterile reconstituted formulation provides a solubilized or dissolved amount of fluticasone that is at least about 75% of the amount of lyophilized fluticasone present prior to reconstitution and/or a solubilized or dissolved amount of salmeterol that is at least about 75% of the amount of the lyophilized salmeterol present prior to reconstitution as determined by HPLC after about 2 minutes of dissolution. In yet further or additional embodiments, the sterile reconstituted formulation provides a solubilized or dissolved amount of fluticasone that is about 90% to about 100% of the amount of lyophilized fluticasone present prior to reconstitution or a solubilized and/or dissolved amount of salmeterol that is at least about 90% to about 100% of the amount of the lyophilized salmeterol present prior to reconstitution as determined by HPLC after about 2 minutes of dissolution.

In one embodiment, provided herein is a sterile formulation that has been reconstituted from a lyophilized composition that comprised fluticasone or a pharmaceutically acceptable salt thereof; salmeterol or a pharmaceutically acceptable salt thereof; a bulking agent; and a non-ionic surfactant wherein the non-ionic surfactant was present in the lyophile composition prior to reconstitution in an amount of about 4% to about 5% of the total combined dry weight of the lyophile. In certain embodiments, the reconstituted sterile formulation is formulated for subcutaneous administration. In further or additional embodiments, the reconstituted sterile formulation is formed by dissolving a lyophile comprising salmeterol and fluticasone in a pharmaceutically acceptable diluent. In some embodiments, the dissolution occurs in less than about 2 minutes. In further or additional embodiments, the dissolution occurs in about 1 minute. In yet other embodiments, the dissolution occurs in less than about 1 minute. In one embodiment, the dissolution results in a clear solution.

Therapeutic and Cosmetic Methods of Using Fluticasone and Salmeterol

In a fifteenth aspect, provided herein are cosmetic or therapeutic methods comprising administering or providing, including for example by subcutaneous administration, to a human a sterile fluticasone and salmeterol formulation that has been reconstituted from a lyophilized composition that comprised fluticasone or a pharmaceutically acceptable salt thereof; salmeterol or a pharmaceutically acceptable salt thereof; a bulking agent; and a non-ionic surfactant. In some embodiments, the lyophilized composition further comprised an anti-oxidant. In some embodiments, the lyophilized sterile composition further comprises an anti-oxidant and/or a buffering agent. In some embodiments, the lyophile further comprises a buffering agent. In further or additional embodiments, the buffering agent is citric acid or sodium citrate dihydrate. In one embodiment, the buffering agent is citric acid. In another embodiment, the buffering agent is citrate dihydrate.

In some embodiments, the reconstituted sterile formulation further comprises a pharmaceutically acceptable diluent that is selected from Sterile Water for Injection, 0.9% sodium chloride solution, or 5% dextrose solution. In one embodiment, the pharmaceutically acceptable diluent is Sterile Water for Injection. In some embodiments, the method is therapeutic. In further or additional embodiments, the method is cosmetic.

In some embodiments, the reconstituted sterile salmeterol and fluticasone formulation is used (for example by provision or administration) to treat an indication selected from the group consisting of abdominal adiposity, regional adiposity, and exophthalmos due to thyroid eye disease. In certain embodiments, the reconstituted sterile salmeterol and fluticasone formulation is administered or provided subcutaneously. In some embodiments, the reconstituted sterile salmeterol and fluticasone formulation is administered or provided to the human subcutaneously as a peri-orbital or intra-orbital injection. In some embodiments, the reconstituted sterile salmeterol and fluticasone formulation is administered or provided to the human subcutaneously to an abdominal region or an ophthalmic region. In further or additional embodiments, provided is a reconstituted salmeterol and fluticasone formulation wherein the formulation is administered or provided to the human in the inside region of the knees, the middle to upper area of the upper arm (including the tricep area), the submental area (including the area under the chin, for example the wattle (which is understood to refer to the fleshy fold of skin in the submental area of a patient)), the abdomen, the hips, the inner thigh, the outer thigh, the buttocks, the lower back, the upper back or the chest.

In further embodiments, provided herein is a cosmetic method comprising administering or providing, including for example by subcutaneous administration, to a human a sterile salmeterol and fluticasone formulation that has been reconstituted from a lyophilized composition that comprised lyophilized salmeterol or a pharmaceutically acceptable salt thereof, lyophilized fluticasone or a pharmaceutically salt thereof, a bulking agent and a non-ionic surfactant. In some embodiments, the lyophilized composition further comprised an anti-oxidant.

In some embodiments, the reconstituted sterile salmeterol and fluticasone formulation is provided cosmetically to the human to affect a shape, contour, or appearance of the human body. In further or additional embodiments, the shape, contour, or appearance is in a region of the body (e.g., the abdominal region) or the eye region of the human. In certain embodiments, the formulation is administered or provided to the human subcutaneously as a peri-orbital or intra-orbital injection. In some embodiments, the sterile reconstituted fluticasone and salmeterol formulation is administered or provided to the human subcutaneously to an abdominal region or an ophthalmic region of a human.

Methods of Manufacturing Fluticasone and Salmeterol Lyophile

In a sixteenth aspect, provided herein are methods of preparing a lyophilized sterile composition comprising: (i) solubilizing fluticasone or a pharmaceutically acceptable salt thereof, salmeterol or a pharmaceutically acceptable salt thereof, a bulking agent, a non-ionic surfactant (and optionally an anti-oxidant) with a solvent or co-solvent to form a bulk solution; (ii) sterilizing the bulk solution; and (iii) lyophilizing the sterilized bulk solution to provide a lyophilized sterile composition. In some embodiments, solubilization step further comprises solubilizing an anti-oxidant and/or a buffering agent. In some embodiments, the solubilization step further comprises solubilizing a buffering agent. In further or additional embodiments, the buffering agent is citric acid or sodium citrate dihydrate. In one embodiment, the buffering agent is citric acid. In another embodiment, the buffering agent is citrate dihydrate.

In some embodiments, the solvent or co-solvent is selected from tert-butyl alcohol, n-butanol, ethanol, iso-propyl alcohol, dimethyl sulfone, chlorobutanol, Sterile Water for Injection, 0.9% sodium chloride solution, 5% dextrose solution, or mixtures thereof. In certain embodiments, the methods described herein further comprise sterilizing the bulk solution comprises passing the bulk solution through a filter. In one embodiment, the filter is a 0.2 micron filter. In further or additional embodiments, the bulk solution has a glass transition temperature of about −35° C. to about −25° C. In some embodiments, the lyophilized fluticasone and/or salmeterol is in an amorphous phase. In still further or additional embodiments, the non-ionic surfactant within the lyophilized composition is present in an amount of about 5.0% by weight of the total combined dry weight of the lyophile.

Methods of Reconstituting a Fluticasone and Salmeterol Lyophile

In a seventeenth aspect, provided herein are methods of preparing a formulation that is suitable for subcutaneous injection comprising the step of contacting with a pharmaceutically acceptable diluent or carrier a lyophilized material comprising: (a) lyophilized fluticasone or a pharmaceutically acceptable salt thereof; (b) lyophilized salmeterol or a pharmaceutically acceptable salt thereof; (c) a bulking agent; and (d) a non-ionic surfactant. In some embodiments, the lyophilized material further comprises an anti-oxidant. In some embodiments, the lyophilized material further comprises an anti-oxidant and/or a buffering agent. In some embodiments, the lyophile material further comprises a buffering agent. In further or additional embodiments, the buffering agent is citric acid or sodium citrate dihydrate. In one embodiment, the buffering agent is citric acid. In another embodiment, the buffering agent is citrate dihydrate.

In some embodiments, the reconstituted sterile fluticasone and salmeterol formulation further comprises a pharmaceutically acceptable diluent or carrier that is selected from Sterile Water for Injection, 0.9% sodium chloride solution, or a 5% dextrose solution. In some embodiments, the pharmaceutically acceptable diluent or carrier is Sterile Water for Injection. In certain embodiments, the non-ionic surfactant is present in the lyophile fluticasone composition is in an amount of about 4% to about 5% of the total combined dry weight of the lyophile. In yet further or additional embodiments, the method provides a solubilized or dissolved amount of fluticasone in the formulation that is about 90% to about 100% of the amount of lyophilized fluticasone present in the composition prior to reconstitution or a solubilized or dissolved amount of salmeterol in the formulation that is about 90% to about 100% of the amount of lyophilized salmeterol present in the composition prior to reconstitution as determined by HPLC after about 2 minutes of dissolution.

Kits for Therapeutic and Cosmetic Use

In a seventeenth aspect, provided herein are kits for therapeutic and cosmetic use. In one embodiment, provided is a kit comprising: (a) a vial comprising lyophilized fluticasone and/or lyophilized salmeterol; (b) a vial comprising a pharmaceutically acceptable diluent or carrier; (c) an injector; and (d) instructions for reconstitution of the lyophilized material and optionally administration to a human. In some embodiments, the kit comprises a vial that comprises a lyophilized material that comprises fluticasone. In further or additional embodiments, the kit comprises a vial that comprises a lyophilized material that comprises salmeterol. In further or additional embodiments, provided is a kit that contains a single vial that comprises a lyophilized material that comprises lyophilized fluticasone and lyophilized salmeterol. In still further or additional embodiments, provided is a kit that contains a pharmaceutically acceptable diluent or carrier that is selected from Sterile Water for Injection, 0.9% sodium chloride solution, or 5% dextrose solution. In yet further or additional embodiments, provided is a kit that contains an injector wherein the injector contains a needle, is needleless, or comprises a subcutaneous applicator.

### BRIEF DESCRIPTION OF THE DRAWINGS

The features of the embodiments described herein are set forth with particularity in the appended claims. A better understanding of the features and advantages presently described herein will be obtained by reference to the following detailed description that sets forth illustrative embodiments, in which the principles are utilized, and the accompanying drawings of which:

FIG. 1 shows an illustrative process schematic for preparation of a fluticasone propionate lyophile.

FIG. 2 shows an illustrative process schematic for preparation of a salmeterol xinafoate lyophile.

FIG. 3 shows an illustrative process schematic for preparation of a fluticasone propionate and salmeterol xinafoate lyophile.

### DETAILED DESCRIPTION OF THE INVENTION

Traditionally, suspension forms have been used to deliver various therapeutic agents for the treatment of different conditions. They are subject, however, to physical instability by flocculation and/or aggregation. Suspensions can affect both the ability of the therapeutic agent in the medicament to be dispersed at the intended site of treatment and its bioavailability once administered. Fluticasone propionate is the approved name for S-fluoromethyl-6α,9α-difluoro-11β-hydroxy-16α-methyl-17α-propionyloxy-3-oxandrosta-1,4-diene-17β-carbothioate, a corticosteroid known to exhibit topical anti-inflammatory activity. In aerosol form, fluticasone propionate is conventionally prepared by micronization. Fluticasone propionate also forms a suspension when mixed with certain solvents. Effective topical treatment of a condition using fluticasone is limited by the ability of fluticasone contained in a powder or suspension to be dispersed effectively across the site of treatment. This limitation makes treatment of some conditions with fluticasone, impractical. Further, while the use of solution forms containing a therapeutic agent are desirable, the therapeutic agent may not be as effective because therapeutic agents are known to break down if the therapeutic agent has been in solution for an extended period of time.

Described herein are lyophilized forms of a composition comprising fluticasone or salmeterol or a combination thereof which, when reconstituted in a suitable solvent, provides a non-suspension (e.g., solubilized) form which may be aseptically sterilized by passing the solution through a suitable 0.2 micron filter and subsequently administered for parenteral (e.g., subcutaneous, peri-orbital, intra-orbital, and intramuscular) administration. The reconstituted non-suspension form provides a clear solution which is, in other embodiments, dispersed to the intended site of treatment and is more readily bioavailable once administered. Further, the lyophilized forms of the compositions described herein provide for a substantially more stable form, which, when needed, can be reconstituted in an acceptable solvent system, such as by way of example only, Sterile Water for Injection to provide a non-suspension injectable form immediately prior to administration. Further, the embodiments described herein provide a reconstituted form within a relatively short period of time, such as by way of example only, 20 seconds by merely shaking the vial containing the lyophile cake formulation and the acceptable solvent or carrier. Such stable forms as described herein are, in other embodiments, stable at various temperatures for extended periods of time. Further, the clear solution permits aseptic filtration and removes the need for the requirement of other sterilization techniques such as terminal sterilization which would compromise the salmeterol and/or fluticasone drug product stability.

The lyophilized compositions and reconstituted formulations described herein provide numerous advantages over prior art compositions and formulations. For example, in some embodiments, the active ingredient(s) of the compositions and formulations provided herein standing alone are poorly soluble in aqueous solutions. As discussed in more detail herein, co-solvents, including lyophilization solvents, are provided. As described herein, in certain embodiments, co-solvents are utilized to solubilize the poorly water soluble active ingredient(s) of the formulations and compositions described herein so that they can be aseptically filtered. Bulking agents are also described. Bulking agents, in some embodiments, are utilized to facilitate the lyophilization of the poorly soluble active ingredient(s) (including amorphous and crystalline forms). Further described herein are non-ionic surfactants. In certain embodiments, non-ionic surfactants are utilized to facilitate later reconstitution of a lyophilized composition that comprises poorly water soluble active ingredient(s) (including fluticasone and salmeterol). For example, in certain situations, a non-ionic surfactant is necessary to force into solution the lyophilized hydrophobic active ingredient(s) in the presence of polar solvents.

Described herein, in certain embodiments, are lyophile fluticasone compositions comprising lyophilized fluticasone or a pharmaceutically acceptable salt thereof, a bulking agent and a non-ionic surfactant. In further or additional embodiments, provided are sterile fluticasone formulations that have been reconstituted from a lyophilized composition that comprised lyophilized fluticasone or a pharmaceutically acceptable salt thereof, a bulking agent and a non-ionic surfactant. In further or additional embodiments, provided are cosmetic and/or therapeutic methods comprising subcutaneously administering or providing to a human a reconstituted sterile fluticasone formulation that, prior to reconstitution, comprised lyophilized fluticasone or a pharmaceutically acceptable salt thereof, a bulking agent and a non-ionic surfactant. Also provided herein are methods of preparing a lyophilized sterile fluticasone composition comprising: (i) solubilizing fluticasone or a pharmaceutically acceptable salt thereof; a bulking agent, and a non-ionic surfactant with a solvent or co-solvent to form a bulk solution; (ii) sterilizing the bulk solution; and (iii) lyophilizing the sterilized bulk solution to provide a lyophilized sterile fluticasone composition. In still further or additional embodiments, provided are reconstitution methods of preparing a fluticasone formulation that is suitable for subcutaneous injection comprising the step of contacting a lyophilized sterile fluticasone composition that further comprises a bulking agent and a non-ionic surfactant with a pharmaceutically acceptable diluent or carrier.

Also described herein, in certain embodiments, are lyophile salmeterol compositions comprising lyophilized salmeterol or a pharmaceutically acceptable salt thereof and a bulking agent. In some embodiments, the salmeterol lyophile further comprises a non-ionic surfactant, an anti-oxidant, and/or a buffering agent. In some embodiments, provided are sterile salmeterol formulations that have been reconstituted from a lyophilized composition that comprised lyophilized salmeterol or a pharmaceutically acceptable salt thereof and a bulking agent. In some embodiments, further provided are cosmetic and/or therapeutic methods comprising subcutaneously administering or providing to a human a reconstituted sterile salmeterol formulation that, prior to reconstitution, comprised lyophilized salmeterol or a pharmaceutically acceptable salt thereof and a bulking agent. In some embodiments, provided is a method of preparing a lyophilized sterile salmeterol composition comprising: (i) solubilizing salmeterol or a pharmaceutically acceptable salt thereof and a bulking agent and optionally a buffering agent with a solvent or co-solvent to form a bulk solution; (ii) sterilizing the bulk solution; and (iii) lyophilizing the sterilized bulk solution to provide a lyophilized sterile salmeterol composition. In certain embodiments, provided is a reconstitution method of preparing a salmeterol formulation that is suitable for subcutaneous injection comprising the step of contacting a lyophilized sterile salmeterol composition that further comprises a bulking agent with a pharmaceutically acceptable diluent or carrier.

Further described herein, in certain embodiments, are lyophile compositions comprising: (a) a lyophilized fluticasone or a pharmaceutically acceptable salt thereof; (b) a lyophilized salmeterol or a pharmaceutically acceptable salt thereof; (c) a bulking agent; and (d) a non-ionic surfactant. In some embodiments, provided are sterile formulations that have been reconstituted from a lyophilized composition that comprised fluticasone or a pharmaceutically acceptable salt thereof; salmeterol or a pharmaceutically acceptable salt thereof; a bulking agent; and a non-ionic surfactant. In some embodiments, provided is a cosmetic and/or therapeutic method comprising subcutaneously administering or providing to a human a reconstituted sterile formulation that, prior to reconstitution, comprised fluticasone or a pharmaceutically acceptable salt thereof; salmeterol or a pharmaceutically acceptable salt thereof; a bulking agent; and a non-ionic surfactant. Further provided herein are methods of preparing a lyophilized sterile composition comprising: (i) solubilizing fluticasone or a pharmaceutically acceptable salt thereof; salmeterol or a pharmaceutically acceptable salt thereof, a bulking agent, a non-ionic surfactant, and an anti-oxidant with a solvent or co-solvent to form a bulk solution; (ii) sterilizing the bulk solution; and (iii) lyophilizing the sterilized bulk solution to provide a lyophilized sterile composition. In some embodiments, provided is a reconstitution method of preparing a formulation that is suitable for subcutaneous injection comprising the step of contacting with a pharmaceutically acceptable diluent or carrier a lyophilized material comprising: (a) fluticasone or a pharmaceutically acceptable salt thereof; (b) salmeterol or a pharmaceutically acceptable salt thereof; (c) a bulking agent; and (d) a non-ionic surfactant. In still further embodiments, provided is a kit comprising: (a) a vial comprising lyophilized fluticasone or lyophilized salmeterol; (b) a vial comprising a pharmaceutically acceptable diluent or carrier; (c) an injector; and (d) instructions for reconstitution of the lyophilized material and optionally administration or provision to a human.

“About” as used herein when referring to a measurable value such as an amount, a temporal duration, and the like, is meant to encompass variations of +/−20% or +/−10%, or in other embodiments, +/−5%, or in further embodiments, +/−1%, or in yet further embodiments, +/−0.1% from the specified value, as such variations are appropriate to perform the disclosed methods or to manufacture and/or prepare the desired composition or formulation.

Provided herein, in certain embodiments, are weights of agents (including fluticasone and salts thereof, salmeterol and salts thereof, and combinations of them) in lyophile compositions (expressed as weight/gram) and reconstituted formulations (expressed in weight/milliliter). With respect to the lyophile compositions, the weights expressed are relative to the total dry weight of the composition. For example, in one embodiment, provided is a sterile lyophilized fluticasone composition comprising about 150 micrograms/gram of lyophilized fluticasone. Thus, in this embodiment, provided is about 150 micrograms of lyophilized fluticasone per 1 gram of the total dry weight of the lyophile composition. With respect to reconstituted formulations, the weights expressed are with respect to the total volume of the reconstituted formulation. As an example, in one embodiment, provided is a reconstituted sterile fluticasone formulation comprising reconstituted fluticasone that is present in an amount of about 1 microgram/milliliter. As such, in this embodiment provided is about 1 microgram of reconstituted fluticasone per 1 milliliter of the total volume of the reconstituted formulation.

Provided herein, in certain embodiments, are therapeutic and/or cosmetic methods and uses including the administration or provision of (1) fluticasone and salts thereof, (2) salmeterol and salts thereof, and (3) combinations of them. It is understood that, in embodiments where the administration or provision of more than one agent is provided, the administration or provision includes concomitant (e.g., as part of the same formulation), sequential (e.g., separately but one agent administered after the other within an acceptable period of time), and concurrent (e.g., at the same time but the agents are, e.g., in different formulations).

Unless defined otherwise, all technical and scientific terms used herein have the same meaning as commonly understood in the art.

### Bulk Solution

Described herein are lyophilized compositions prepared by lyophilizing bulk solutions comprising an active ingredient, such as by way of example only, salmeterol, fluticasone or their pharmaceutically acceptable salts, or a combination thereof. In one embodiment, the active ingredient described herein to be lyophilized is substantially homogeneously mixed with at least one non-ionic surfactant and at least one bulking agent in an aqueous and/or organic solvent system. The term “substantially homogeneous” is defined as the components are substantially uniformly dispersed in each other, for example, as in a solution, or bulk solution.

Fluticasone is a synthetic glucocorticosteroid and has the chemical structure:

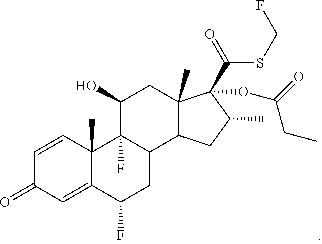

Pharmaceutically acceptable salts of fluticasone include but are not limited to the propionate and furoate salts.

Salmeterol is a long acting, selective, and liophilic beta adrenergic receptor agonist and has the chemical structure:

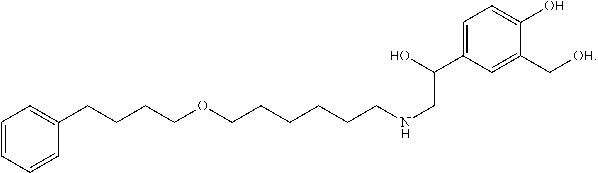

An illustrative pharmaceutically acceptable salt of salmeterol includes the xinafoate salt, shown below:

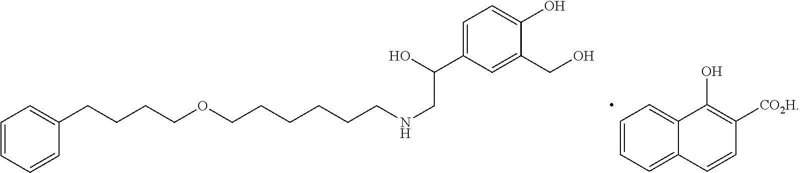

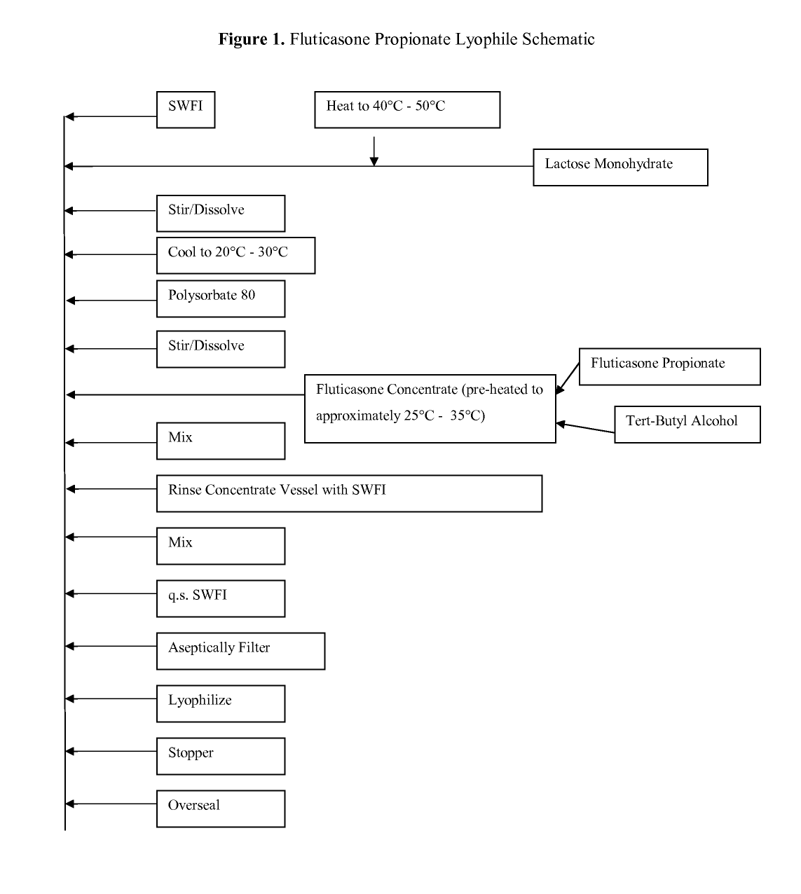

As described above, the lyophiles presented herein are formed by lyophilization of the bulk solution. The bulk solution contains among other components, fluticasone and/or salmeterol or their pharmaceutically acceptable salts thereof and a suitable organic solvent system. The organic solvent system, in some embodiments, contains a sufficient amount of organic solvent to solubilize the active ingredient, such as for example, fluticasone propionate or salmeterol xinafoate or a combination thereof, to form a substantially homogenous liquid mixture (FIGS. 1, 2, and 3 refer to the substantially homogenous liquid mixture as a “concentrate.”) In some embodiments, the solvent, such as a lower alcohol and therapeutic agent, such as fluticasone or salmeterol or a pharmaceutically acceptable salt thereof, are combined at a temperature sufficient to dissolve the therapeutic agent and ensure that the solvent is in a liquid state, and then mixed by vortexing to form a substantially homogenous liquid mixture. In some embodiments, the mixing takes anywhere from about 10 minutes to about 3 hours. In certain embodiments, the mixing occurs for a period of about 15 minutes to about 2 hours. In further or additional embodiments, the mixing occurs for a period of time of about 30 minutes to about 90 minutes. In one embodiment, the mixing occurs for about 60 minutes. Optionally, sonication, the use of a static mixer, blade mixer, homogenizer and the like, will in some embodiments, follow to ensure complete dissolution.

In one embodiment, the organic solvent comprises lower oxyhydrocarbons, lower halohydrocarbons, lower haloxyhydrocarbons, lower sulfoxyhydrocarbons, lower cyclohydrocarbons and combinations thereof. In one embodiment, the solvent system includes, but is not limited to, tert-butyl alcohol, isopropyl alcohol, methanol, ethanol, acetone, acetonitrile, cyclohexane, chlorobutanol, dimethylsulfoxide, hexanol, benzyl alcohol, acetic acid, pentanol, n-propanol, n-butanol, methyl ethyl ketone, dimethyl sulfone, chlorobutanol and combinations thereof. In other embodiments, the solvent for use in preparing the lyophilized compositions described herein includes lower alkanols, such as by way of example only, ethanol, isopropyl alcohol and tert-butyl alcohol. In other embodiments, the solvent is tert-butyl alcohol. In still further or additional embodiments, the solvent is ethanol.

The term “lower oxyhydrocarbons” as referred to herein means compounds possessing hydrocarbyl radicals and oxygen atoms having from 1 to 8 carbon atoms and from 1 to 4 oxygen atoms. Examples of “lower oxyhydrocarbons” include, but are not limited to, lower alkanols, lower ketones, lower carboxylic acids, lower carboxylic esters, lower carbonates, and the like.

In some embodiments, the organic solvent is from about 0.1% by volume to about 30% by volume of the bulk solution. In other embodiments, the organic solvent is from about 0.5% by volume to about 15% by volume of the bulk solution. In further embodiments, the organic solvent is about 0.1% to about 1% by volume of the bulk solution. In other embodiments, the organic solvent is less than about 10% by volume of the bulk solution.

In other embodiments, the bulk solution forms a substantially homogenous mixture having a temperature in the range of about −10° C. to about 50° C.

In further embodiments, the bulk solution comprises a bulking agent. In some embodiments, the bulking agent is selected from lactose, DL-alanine, glucose, D(+)trehalose dihydrate, sucrose, maltose, D(+)raffinose pentahydrate, sodium saccharin, starches, modified celluloses, dextrins, dextrans, glycine, sodium chloride, calcium carbonate, sodium tartrate and calcium lactate. In other embodiments, the bulking agent is lactose monohydrate.

In some embodiments, the bulking agent is dissolved in a pharmaceutically acceptable carrier, such as for example, Sterile Water for Injection, and heated to a temperature sufficient to allow dissolution. In other embodiments, the bulking agent mixture is cooled to room temperature and a non-ionic surfactant is added.

Non-ionic surfactants include: (N,N-Bis[3-(D-gluconamido)propyl]cholamide); Bis(polyethylene glycol bis[imidazoyl carbonyl]); Polyoxyethyleneglycol dodecyl ether; Polyoxyethylenglyceroltriricinoleate 35; decaethylene glycol monododecyl ether; N-decanoyl-N-methylglucamine; n-decyl α-D-glucopyranoside; decyl β-D-maltopyranoside; n-dodecanoyl-N-methylglucamide; n-dodecyl α-D-maltoside; n-dodecyl β-D-maltoside; n-hexadecyl β-D-maltoside; heptaethylene glycol monodecyl ether; heptaethylene glycol monododecyl ether; heptaethylene glycol monotetradecyl ether; hexaethylene glycol monododecyl ether; hexaethylene glycol monohexadecyl ether; hexaethylene glycol monooctadecyl ether; hexaethylene glycol monotetradecyl ether; octylphenyl-polyethylene glycol; methyl-6-O—(N-heptylcarbamoyl)-α-D-glucopyranoside; nonaethylene glycol monododecyl ether; N-nonanoyl-N-methylglucamine; N-nonanoyl-N-methylglucamine; octaethylene glycol monodecyl ether; octaethylene glycol monododecyl ether; octaethylene glycol monohexadecyl ether; octaethylene glycol monooctadecyl ether; octaethylene glycol monotetradecyl ether; octyl-β-D-glucopyranoside; pentaethylene glycol monodecyl ether; pentaethylene glycol monododecyl ether; pentaethylene glycol monohexadecyl ether; pentaethylene glycol monohexyl ether; pentaethylene glycol monooctadecyl ether; pentaethylene glycol monooctyl ether; polyethylene glycol diglycidyl ether; polyethylene glycol ether W-1; polyoxyethylene 10 tridecyl ether; polyoxyethylene 100 stearate; polyoxyethylene 20 isohexadecyl ether; polyoxyethylene 20 oleyl ether; polyoxyethylene 40 stearate; polyoxyethylene 50 stearate; polyoxyethylene 8 stearate, polyoxyethylene bis(imidazolyl carbonyl), polyoxyethylene 25 propylene glycol stearate; saponin; sorbitan laurate, sorbitan monopalmitate, sorbitan stearate, sorbitan tristearate, sorbitan oleate, sorbitan trioleate; polyethyelene glycol trimethylnonyl ether, nonylphenol ethoxylate; tetradecyl-β-D-maltoside; tetraethylene glycol monodecyl ether, tetraethylene glycol monododecyl ether, tetraethylene glycol monotetradecyl ether; triethylene glycol monodecyl ether, triethylene glycol monododecyl ether, triethylene glycol monohexadecyl ether, triethylene glycol monooctyl ether, triethylene glycol monotetradecyl ether; octoxynol-9; octylphenol ethoxylate; polysorbate 20, polysorbate 21, polysorbate 40, polysorbate 60, polysorbate 61, polysorbate 65, polysorbate 80, polysorbate 81, polysorbate 85; tyloxapol; and n-undecyl β-D-glucopyranoside. In one embodiment, the non-ionic surfactant used in the bulk solution is polysorbate 80.

In further embodiments, the salmeterol concentrate is added to the bulking agent/non-ionic surfactant mixture and mixed using methods described herein.

In further embodiments, an anti-oxidant is added to the bulk solution. In yet another embodiment, the anti-oxidant is ascorbic acid.

In a further embodiment, a buffering agent is added to the bulk solution. In yet another embodiment, the buffering agent is citric acid monohydrate and/or sodium citrate dihydrate.

### Filtration Methods

Provided herein are mixtures of fluticasone, salmeterol and/or combinations thereof, combined to form the bulk solution/liquid mixture, wherein the liquid mixture is sterilized. In one embodiment, the substantially homogenous bulk solution/liquid mixture is filter sterilized before lyophilization. In another embodiment, the filtration sterilization step is performed to remove any contaminants of, e.g., bioburden, in the bulk solution/liquid mixture and provide a relatively/substantially sterile solution. “Sterilized” or “substantially sterile” is defined by a process which all viable forms of microorganisms are removed or destroyed, based upon a probability function.

Suitable filters include sterile filters that are compatible with organic solvents. A suitable filler includes, for example a cellulose filter. In one embodiment is a method of sterilizing the bulk solution comprising the use of a poly(tetrafluoroethylene) (PTFE) filter as it is generally compatible with alcohols and related organic or organic-aqueous media, including tert-butyl alcohol and/or ethanol. In another embodiment, the filter used in the methods described herein is composed of poly(ether-sulfone). In further embodiments, the filters are selected from filters composed of polyvinylidene fluoride, polypropylene and nylon.

In some embodiments, the porosity of the filter is from about 0.1 to about 0.5 microns. In another embodiment, the porosity of the filter used in preparing sterile lyophilized compositions described herein is about 0.2 microns. The size and surface area of the filter will determine the volume of the liquid mixture that is passed through the filter. In other embodiments, following filtration, the compositions are placed in suitable lyophilization vials that, in further embodiments are single dose formulations following reconstitution. In yet other embodiments, the vials are generally depyrogenated and sterilized and in further embodiments range from about 1 mL to 3 mL vials. In other embodiments, the lyophilized compositions described herein are terminally sterilized, e.g., sterilized after lyophilization has occurred. In yet other embodiments, terminal sterilization is via gamma irradiation, or e-beam sterilization.

### Lyophilization Methods

Lyophilization is the technical name for a process often referred to as “freeze-drying.” In this process, an aqueous mixture or suspension is frozen into a solid, then it is generally subjected to a vacuum for a substantial period of time. The vacuum causes the water molecules to sublimate.

The methods described herein include the step of lyophilizing the active ingredient, such as for example, salmeterol or fluticasone, or a combination of both, or their pharmaceutically acceptable salts thereof. In one embodiment, lyophilization occurs after sterilization.

In one embodiment, during the lyophilization process, the solvent system used, such as by way of example only, tert-butyl alcohol and Sterile Water for Injection is substantially removed by sublimation. In another embodiment, less than about 5% residual solvent remains after lyophilization; in other embodiments, less than about 3% remains; in yet other embodiments, less than about 2% remains; in further embodiments, less than about 1% or about 0.1% remains.

In one embodiment, the lyophilization process comprises the steps of (1) placing the sample to be lyophilized (salmeterol, fluticasone or a combination thereof, or their pharmaceutically acceptable salts thereof) in a suitable vial and placing the vial into a lyophilization chamber and lowering the shelf temperature to about −30° C. to about −50° C. at atmospheric pressure; (2) holding the shelf temperature at the temperature range described above until the temperature of the sample is about −30° C. to about −50° C.; (3) raising the temperature to about −10° C. to about −20° C. to anneal the lyophile for about 1 to 2 hours; (4) lowering the shelf temperature to about −30° C. to about −50° C. and reducing the pressure of the system to about 50 mTorr to about 100 mTorr; and holding until sublimation of the solvent system is substantially complete. The temperature of the product should be below about −25° C. to about −28° C. to avoid cake collapse; (5) increasing the temperature to about 30° C. to about 50° C.; and (6) allowing the samples to reach a temperature of about 20° C. to about 30° C. for an amount of time to remove bound water or solvent levels; (7) back-filling vials with nitrogen or appropriate gas after which the vials are aseptically sealed. Table 7 describes, in one embodiment, the lyophilization cycle for lyophilizing a bulk solution comprising fluticasone or salmeterol or their pharmaceutically acceptable salts or a combination thereof. In one embodiment, the process requires a step-wise lowering or increasing of the temperature of the system, such as, at a rate of 0.5° C. per minute up to about 1° C. per minute to ensure proper and substantially complete sublimation. The lyophilization step provides a composition comprising an active compound, such as for example, salmeterol or fluticasone, or their pharmaceutically acceptable salts or a combination thereof, that can be stored at room temperature for extended periods of time. Additionally, the lyophilized compositions described herein are stable for a period of at least 4 weeks at a temperature of about 0° C. to about 50° C. In some embodiments, the lyophilized compositions are stable from at least about 3 months to at least about 5 years at a temperature of about 0° C. to about 50° C. In certain embodiments, the lyophilized compositions are stable for a period of at least about 4 months to at least about 4 years at a temperature of about 0° C. to about 50° C. In still further or additional embodiments, the lyophilized compositions are stable for a period of at least about 6 months to at least about 2 years at a temperature of about 0° C. to about 50° C. In some embodiments, the lyophilized compositions are stable for at least about 3 months, at least about 6 months, at least about 1 year, at least about 2 years, at least about 3 years, at least about 4 years, or at least about 5 years at a temperature of about 0° C. to about 50° C. In other embodiments, the lyophilized compositions described herein are in the form of a cake or free flowing powder. In other embodiment, the lyophilized composition is a cake.

### Reconstitution

In some embodiments, the lyophilized compositions described herein readily reconstitute once contacted with a sufficient amount of a pharmaceutically acceptable carrier. For example, in some embodiments, the lyophilized composition is mixed in the vial it is contained in, e.g., shaken for about 1 to about 3 minutes, with a pharmaceutically acceptable carrier, such as, Sterile Water for Injection, 0.9% sodium chloride solution, or 5% dextrose solution to provide a reconstituted composition suitable for subcutaneous, peri-orbital, intra-orbital, and intramuscular injection. In one embodiment, the lyophilized composition is reconstituted in a relatively short period of time, such as for example, less than 1 minute, less than 30 seconds, and in other embodiments, about 20 seconds. In certain embodiments, the lyophilized compositions reconstitute in a time of less than 2 minutes. These short reconstitution times provide an advantage in that the therapeutic agent has not decomposed from exposure in a solution for an extended period of time prior to administration. In one embodiment, the reconstituted composition is suitable for subcutaneous administration, such as for example, subcutaneous injection. In another embodiment, the reconstituted form is a non-suspension. In a further embodiment, the reconstituted form is a clear solution and remains substantially clear prior to administration.

An inventive feature of the subject matter described herein is a lyophilized composition (comprising fluticasone, salmeterol, and/or their mixture) that is formulated with a minimal amount of non-ionic surfactant, that is manufactured as a lyophile, and that is amenable to full reconstitution with a carrier or diluents in a short period of time. See, e.g., Example 3 and Table 3.

Accordingly, in one embodiment, provided is a reconstituted lyophile fluticasone formulation that provides a solubilized or dissolved amount of fluticasone that is at least about 50% of the amount of the lyophilized fluticasone present prior to reconstitution as determined by HPLC after about 2 minutes of dissolution. In further or additional embodiments, provided herein is a reconstituted lyophile fluticasone formulation that provides a solubilized or dissolved amount of fluticasone that is at least about 60% of the amount of lyophilized fluticasone present prior to reconstitution as determined by HPLC after about 2 minutes of dissolution. In some embodiments, provided is a reconstituted lyophile fluticasone formulation that provides a solubilized or dissolved amount of fluticasone that is at least about 75% of the amount of lyophilized fluticasone present prior to reconstitution as determined by HPLC after about 2 minutes of dissolution. In further or additional embodiments, provided is a reconstituted lyophile fluticasone formulation that provides a solubilized or dissolved amount of fluticasone that is about 90% to about 100% of the amount of lyophilized fluticasone present prior to reconstitution as determined by HPLC after about 2 minutes of dissolution. In some embodiments, provided is a reconstituted lyophile fluticasone formulation that provides a solubilized or dissolved amount of fluticasone that is about 90% to about 100% of the amount of lyophilized fluticasone present prior to reconstitution as determined by HPLC after about 2 minutes of dissolution wherein the non-ionic surfactant was present in the lyophile fluticasone composition prior to reconstitution in an amount of about 4% to about 5% of the total combined dry weight of the lyophile.

In further or additional embodiments, provided is a reconstituted lyophile salmeterol formulation that provides a solubilized or dissolved amount of salmeterol that is at least about 50% of the amount of the lyophilized salmeterol present prior to reconstitution as determined by HPLC after about 2 minutes of dissolution. In further or additional embodiments, provided herein is a reconstituted lyophile salmeterol formulation that provides a solubilized or dissolved amount of salmeterol that is at least about 60% of the amount of lyophilized salmeterol present prior to reconstitution as determined by HPLC after about 2 minutes of dissolution. In some embodiments, provided is a reconstituted lyophile salmeterol formulation that provides a solubilized or dissolved amount of salmeterol that is at least about 75% of the amount of lyophilized salmeterol present prior to reconstitution as determined by HPLC after about 2 minutes of dissolution. In further or additional embodiments, provided is a reconstituted lyophile salmeterol formulation that provides a solubilized or dissolved amount of salmeterol that is about 90% to about 100% of the amount of lyophilized salmeterol present prior to reconstitution as determined by HPLC after about 2 minutes of dissolution.

In still further or additional embodiments, provided is a reconstituted lyophile fluticasone and salmeterol formulation that provides a solubilized or dissolved amount of fluticasone and/or salmeterol that is at least about 50% of the amount of the lyophilized agent present prior to reconstitution as determined by HPLC after about 2 minutes of dissolution. In further or additional embodiments, provided herein is a reconstituted lyophile fluticasone and salmeterol formulation that provides a solubilized or dissolved amount of fluticasone and/or salmeterol that is at least about 60% of the amount of lyophilized agent present prior to reconstitution as determined by HPLC after about 2 minutes of dissolution. In some embodiments, provided is a reconstituted lyophile fluticasone and salmeterol formulation that provides a solubilized or dissolved amount of fluticasone and/or salmeterol that is at least about 75% of the amount of lyophilized agent present prior to reconstitution as determined by HPLC after about 2 minutes of dissolution. In further or additional embodiments, provided is a reconstituted lyophile fluticasone and salmeterol formulation that provides a solubilized or dissolved amount of fluticasone and/or salmeterol that is about 90% to about 100% of the amount of lyophilized agent present prior to reconstitution as determined by HPLC after about 2 minutes of dissolution. In some embodiments, provided is a reconstituted lyophile fluticasone and salmeterol formulation that provides a solubilized or dissolved amount of fluticasone and/or salmeterol that is about 90% to about 100% of the amount of lyophilized agent present prior to reconstitution as determined by HPLC after about 2 minutes of dissolution wherein the non-ionic surfactant was present in the lyophile salmeterol composition prior to reconstitution in an amount of about 4% to about 5% of the total combined dry weight of the lyophile.

While certain embodiments have been described, these embodiments have been presented by way of example only, and are not intended to limit the scope of the disclosure. The formulations, methods, and systems described herein may be embodied in a variety of other forms. Furthermore, various omissions, substitutions and changes in the form of the formulations, methods, and systems described herein may be made without departing from the spirit of this disclosure. The accompanying claims and their equivalents are intended to cover such forms or modifications.

### EXAMPLES

The following specific examples are to be construed as merely illustrative, and not limitative of the remainder of the disclosure in any way whatsoever. The examples described herein reference and provide non-limiting support to the various embodiments described in the preceding sections.

### Example 1

### Fluticasone Propionate Lyophile

A fluticasone propionate lyophile was prepared by mixing the components in their respective amounts as shown below in Table 1:

TABLE 1

| Component | Weight (mg/g) |
|---|---|
| Fluticasone propionate | 0.2 |
| Lactose monohydrate | 1,000 |
| Polysorbate 80 | 50 |
| Tert butyl alcohol† | 1,000 |
| Sterile Water for Injection† | q.s. |

†Removed during lyophilization

Manufacture of the lyophile formulation of Example 1 is depicted in the schematic of FIG. 1. Briefly, lactose monohydrate was dissolved in Sterile Water for Injection preheated to approximately 40° C.-50° C. After cooling the lactose solution to 20° C.-25° C., polysorbate 80 was added and mixed until homogenous. The fluticasone propionate was dissolved in tert-butyl alcohol solution, preheated to approximately 25° C.-35° C., and added to the aqueous lactose-polysorbate solution and mixed until homogenous. Sterile Water for Injection was added to a target weight. The solution is aseptically filtered through a 0.2 micron filter and subsequently filled into suitable glass vials. The vials are loaded into an appropriate lyophilizer and the water and solvent are removed after running a cycle similar to that described in Table 7. At the conclusion of the cycle the vials are back-filled with an inert gas such as nitrogen prior to stoppering and oversealing.

### Example 2

### Stability of Fluticasone Propionate Lyophile

The methodology for assay potency as described below in Table 4 was utilized in generating the stability data presented in Table 2 after formulations formulated as described above in Table 1 were stored in controlled stability chambers at the specified conditions.

TABLE 2

| Assay (% | Storage Condition | | | |
|---|---|---|---|---|
| Label Claim) | 5° C./ | 25° C./ | 30° C./ | 40° C./ |
| Storage Time | Ambient | 60% RH | 65% RH | 75% RH |
| Initial | 98 | 98 | 98 | 98 |
| 1 month | 96 | 97 | 97 | 95 |
| 2 month | 97 | 98 | 96 | 96 |
| 3 month | 96 | 95 | 94 | 93 |
| 6 month | 99 | 98 | 97 | 100 |

### Example 3

### Reconstitution Assay and Reconstitution Time of Fluticasone Lyophile Formulation

As described herein, an inventive feature of the subject matter described herein is a lyophilized composition (comprising fluticasone, salmeterol, and/or their mixture) that is formulated with a minimal amount of non-ionic surfactant, that is manufactured as a lyophile, and that is amenable to full reconstitution with a carrier or diluent in a short period of time. The potencies and respective reconstitution times of fluticasone propionate with Sterile Water for Injection as measured by HPLC are shown in Table 3. Reconstitution time and assay values were determined by reconstituting the formulations with 1.0 mL of Sterile Water for Injection. The reconstitution time was determined as the time there were no visible particles in solution, in which case the formulation was said to be “clear.” As shown in Table 3, about 100% of the fluticasone propionate in the lyophilized composition was dissolved or solubilized after about 15 seconds of dissolution when 5 mg/g of polysorbate 80 was used in the fluticasone lyophile.

TABLE 3

| | Weight | Weight | Weight | Weight | Weight | Weight |
|---|---|---|---|---|---|---|
| Component | (mg/g) | (mg/g) | (mg/g) | (mg/g) | (mg/g) | (mg/g) |
| Fluticasone | 0.2 | 0.2 | 0.2 | 0.2 | 0.2 | 0.2 |
| Propionate | | | | | | |
| Lactose | 1,000 | 1,000 | 1,000 | 1,000 | 1,000 | 1,000 |
| monohydrate | | | | | | |
| Polysorbate 80 | 50 | 35 | 25 | 0 | 10 | 25 |
| Tert butyl | 1,000 | 1,000 | 1,000 | 1,000 | 2,000 | 2,000 |
| alcohol† | | | | | | |
| Water† | q.s. | q.s. | q.s. | q.s. | q.s. | q.s. |
| Assay (%) | 100% | 60% | 36% | 10% | 21% | 78% |
| Reconstitution | 15 | <60 | PR | PR | PR | <60 |
| Time (seconds) | | | | | | |

†Removed during lyophilization
PR-precipitate present

The methodology for HPLC as described below in Table 4 was utilized in generating the stability data of Example 2 and the reconstitution assay and time data of Example 3.

TABLE 4

| Mobile Phase A | 95% water/5% acetonitrile; 0.05% formic acid | | |
|---|---|---|---|
| Mobile Phase B | 5% water/95% acetonitrile; 0.05% formic acid | | |
| Column | Phenomenex Gemini C-18, 3 mm × 50 mm, | | |
| | 3 μm or equivalent | | |
| Column temperature | 40° C. | | |
| Autosampler temperature | 25° C. | | |
| Detector | UV at wavelength of 228 nm | | |
| Injection volume | 20 μL | | |
| Flow rate | 0.75 mL/minute | | |
| Run time | 15 minutes | | |
| | Time | % | % Mobile |
| | (minutes) | Mobile Phase A | Phase B |
| Gradient Program | 0 | 70 | 30 |
| | 10 | 30 | 70 |
| | 10.1 | 20 | 80 |
| | 11.6 | 20 | 80 |
| | 11.7 | 70 | 30 |
| | 15 | 70 | 30 |

### Example 4

### Salmeterol Xinafoate Lyophile

A salmeterol xinafoate lyophile was prepared by mixing the components in their respective amounts as shown below in Table 5:

TABLE 5

| Component | Weight (mg/g) |
|---|---|
| Salmeterol Xinafoate | 0.1 |
| Lactose monohydrate | 1,000 |
| Ethyl Alcohol (96%)† | 10 μL or weight adjust |
| Sterile Water for Injection† | q.s. |

†Removed during lyophilization

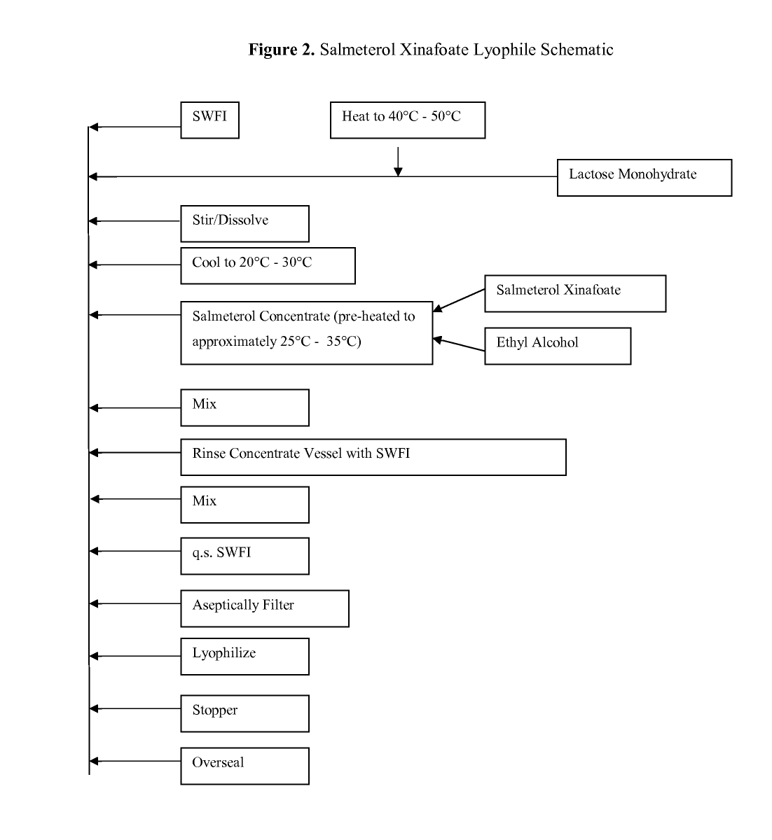

Manufacture of the lyophile composition in Example 4 (and shown in Table 5) is described in the schematic of FIG. 2. The process of preparation used in Example 4 mirrors the process as explained in Example 1.

### Example 5

### Lyophile Combination

A fluticasone propionate and salmeterol xinafoate combination lyophile is prepared by mixing the components in their respective amounts as shown below in Table 6:

TABLE 6

| Component | Weight (mg/g) |
|---|---|
| Fluticasone Propionate | 0.2 |
| Salmeterol Xinafoate | 0.1 |
| Lactose monohydrate | 1,000 |
| Polysorbate 80 | 50 |
| Tert butyl alcohol† | 1,000 |
| Sterile Water for Injection† | q.s. |

†Removed during lyophilization

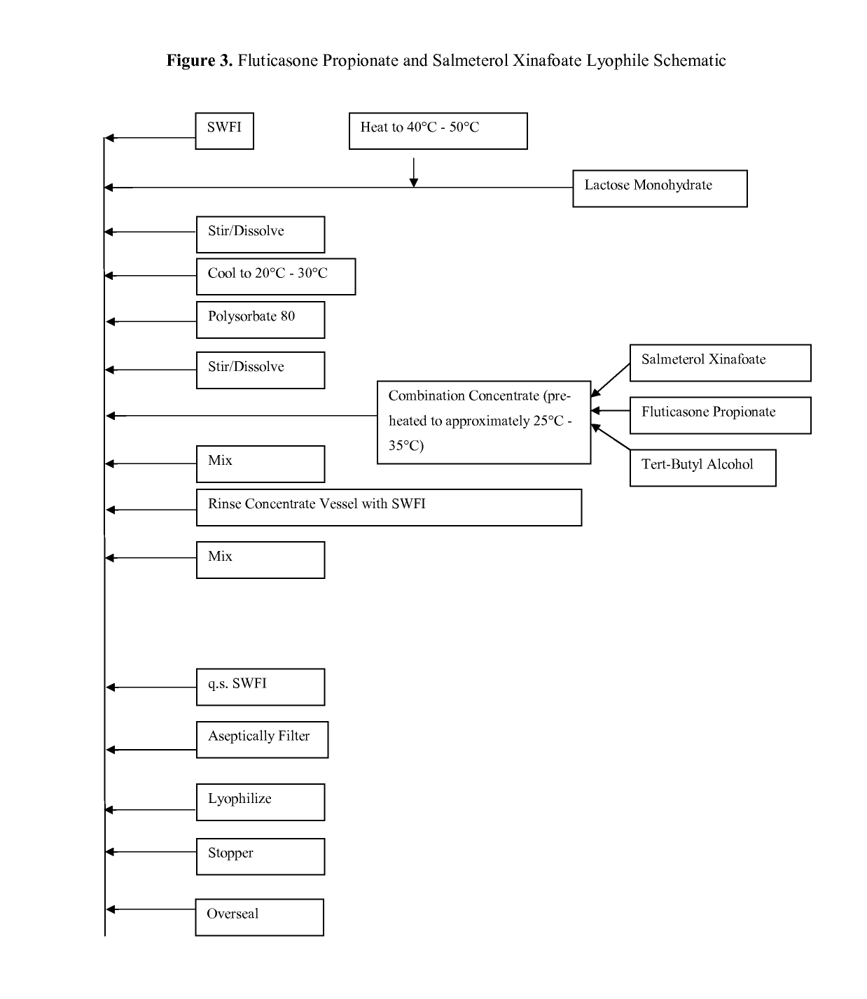

Manufacture of the lyophile composition in Example 5 is described in the schematic of FIG. 3. The process used in Example 5 mirrors the process used in Example 1.

### Example 6

### Lyophilization Cycle

Approximately 1 mL of filtered solution was filled into 3-mL glass vials for each formulation and loaded into a lyophilizer. Formulations were lyophilized according to the lyophilization cycle described below in Table 7.

TABLE 7
Lyophilization Cycle

| | Hold/ | | Time | Pressure | |
|---|---|---|---|---|---|
| Temperature | Ramp | Rate | (minutes) | (mT) | Function |
| −5° C. | Hold | — | 0 | Ambient | Load |
| −40° C. | Ramp | 0.5° C./minute | 70 | Ambient | Freeze |
| −40° C. | Hold | — | 120 | Ambient | Freeze |
| −15° C. | Ramp | 0.5° C./minute | 50 | Ambient | Anneal |
| −15° C. | Hold | — | 120 | Ambient | Anneal |
| −40° C. | Ramp | 0.5° C./minute | 50 | Ambient | Freeze |
| −40° C. | Hold | — | 120 | Ambient | Freeze |
| | | | | | Primary |
| −20° C. | Ramp | 1° C./minute | 20 | 75 | Drying |
| | | | | | Primary |
| −20° C. | Hold | — | 2400 | 75 | Drying |
| | | | | | Secondary |
| 40° C. | Ramp | 2.5° C./min | 150 | 75 | Drying |
| | | | | | Secondary |
| 40° C. | Hold | — | 240 | 75 | Drying |
| 20° C. | Ramp | 2.5° C./min | 50 | Nitrogen/ | Stopper |
| | | | | Ambient | |
| 20° C. | Hold | — | — | Ambient | Unload |

At the conclusion of the lyophilization cycle, vials were stoppered and sealed prior to characterization and analysis.

### Example 7

### Glass Transition Experiment for Bulk Solution

Glass transition (T_{g}) data for bulk solution were generated utilizing Differential Scanning calorimetry. Approximately 10 to 20 mg of bulk solution were pipetted into an aluminum pan and crimped. An empty crimped pan served as the reference. The temperature program used for the DSC analysis was to hold for 1 minute at 25° C., and then cool from 25° C. to −50° C. at 10/minute.

The examples and embodiments described herein are for illustrative purposes only and various modifications or changes are included within the spirit and purview of this application and scope of the appended claims.

What is claimed is:

1. A lyophile fluticasone composition comprising lyophilized fluticasone or a pharmaceutically acceptable salt thereof, a bulking agent and a non-ionic surfactant.

2. A sterile fluticasone formulation that has been reconstituted from a lyophilized composition that comprised lyophilized fluticasone or a pharmaceutically acceptable salt thereof, a bulking agent and a non-ionic surfactant.

3. A cosmetic or therapeutic method comprising subcutaneously administering or providing to a human a reconstituted sterile fluticasone formulation that, prior to reconstitution, comprised lyophilized fluticasone or a pharmaceutically acceptable salt thereof, a bulking agent and a non-ionic surfactant.

4. A method of preparing a lyophilized sterile fluticasone composition comprising:
(i) solubilizing fluticasone or a pharmaceutically acceptable salt thereof, a bulking agent, and a non-ionic surfactant with a solvent or co-solvent to form a bulk solution;
(ii) sterilizing the bulk solution; and
(iii) lyophilizing the sterilized bulk solution to provide a lyophilized sterile fluticasone composition.

5. A reconstitution method of preparing a fluticasone formulation that is suitable for subcutaneous injection comprising the step of contacting a lyophilized sterile fluticasone composition that further comprises a bulking agent and a non-ionic surfactant with a pharmaceutically acceptable diluent or carrier.

6. A lyophile salmeterol composition comprising lyophilized salmeterol or a pharmaceutically acceptable salt thereof and a bulking agent.

7. A sterile salmeterol formulation that has been reconstituted from a lyophilized composition that comprised lyophilized salmeterol or a pharmaceutically acceptable salt thereof and a bulking agent.

8. A cosmetic or therapeutic method comprising subcutaneously administering or providing to a human a reconstituted sterile salmeterol formulation that, prior to reconstitution, comprised lyophilized salmeterol or a pharmaceutically acceptable salt thereof and a bulking agent.

9. A method of preparing a lyophilized sterile salmeterol composition comprising:
(i) solubilizing salmeterol or a pharmaceutically acceptable salt thereof and a bulking agent with a solvent or co-solvent to form a bulk solution;
(ii) sterilizing the bulk solution; and
(iii) lyophilizing the sterilized bulk solution to provide a lyophilized sterile salmeterol composition.

10. A reconstitution method of preparing a salmeterol formulation that is suitable for subcutaneous injection comprising the step of contacting a lyophilized sterile salmeterol composition that further comprises a bulking agent with a pharmaceutically acceptable diluent or carrier.

11. A lyophile composition comprising:
(a) a lyophilized fluticasone or a pharmaceutically acceptable salt thereof;
(b) a lyophilized salmeterol or a pharmaceutically acceptable salt thereof;
(c) a bulking agent; and
(d) a non-ionic surfactant.

12. A sterile formulation that has been reconstituted from a lyophilized composition that comprised fluticasone or a pharmaceutically acceptable salt thereof, salmeterol or a pharmaceutically acceptable salt thereof, a bulking agent, and a non-ionic surfactant.

13. A cosmetic or therapeutic method comprising subcutaneously administering or providing to a human a reconstituted sterile formulation that, prior to reconstitution, comprised fluticasone or a pharmaceutically acceptable salt thereof, salmeterol or a pharmaceutically acceptable salt thereof, a bulking agent, and a non-ionic surfactant.

14. A method of preparing a lyophilized sterile composition comprising:
(i) solubilizing fluticasone or a pharmaceutically acceptable salt thereof, salmeterol or a pharmaceutically acceptable salt thereof, a bulking agent, and a non-ionic surfactant with a solvent or co-solvent to form a bulk solution;
(ii) sterilizing the bulk solution; and
(iii) lyophilizing the sterilized bulk solution to provide a lyophilized sterile composition.

15. A reconstitution method of preparing a formulation that is suitable for subcutaneous injection comprising the step of contacting with a pharmaceutically acceptable diluent or carrier a lyophilized material comprising:
(a) fluticasone or a pharmaceutically acceptable salt thereof;
(b) salmeterol or a pharmaceutically acceptable salt thereof;
(c) a bulking agent; and
(d) a non-ionic surfactant.
